(19) United States
(12) Patent Application Publication
(10) Pub. No.: US 2009/0186832 A1
(43) Pub. Date: Jul. 23, 2009

(54) AMINO ACID PEPTIDE PRO-DRUGS OF PHENOLIC ANALGESICS AND USES THEREOF

(75) Inventors: Richard Franklin, Hampshire (GB); Bernard T. Golding, Newcastle upon Tyne (GB); Robert G. Tyson, Durham (GB)

Correspondence Address: DARBY & DARBY P.C., P.O. BOX 770, Church Street Station, New York, NY, 10008-0770, US

(73) Assignee: SHIRE LLC, Florence, KY (US)

(21) Appl. No.: 12/356,028

(22) Filed: Jan. 19, 2009

Related U.S. Application Data
(60) Provisional application No. 61/022,044, filed on Jan. 18, 2008.
(60) Provisional application No. 61/022,159, filed on Jan. 18, 2008.

(51) Int. Cl. A61K 38/05; C07C 271/50; C07D 223/04; A61K 31/55; C07D 489/08; A61K 31/485; A61K 31/4355

(52) U.S. Cl. 514/19; 560/160; 540/611; 514/212.01; 546/45; 514/282; 514/279

(57) ABSTRACT

Prodrugs of meptazinol and other phenolic analgesics exhibiting low oral bioavailability with amino acids or lower peptides, pharmaceutical compositions containing such prodrugs and a method for providing pain relief with such prodrugs are provided. In addition, the present invention relates to methods for increasing the oral bioavailability of a phenolic analgesic. The method comprises orally administering a phenolic analgesic prodrug, wherein the phenolic analgesic is bound to an amino acid or peptide via a carbamate linkage, to a subject in need thereof. Prodrugs having side chains of valine, leucine, isoleucine and glycine amino acids and mono-, di- and tripeptides thereof are preferred.

### RELATED APPLICATIONS

This application claims the benefit of U.S. Provisional Application Nos. 61/022,044 and 61/022,159, both filed Jan. 18, 2008. These prior applications are hereby incorporated by reference.

### FIELD OF THE INVENTION

The present invention relates to the utilization of amino acid and small peptide prodrugs of meptazinol, oxymorphone, buprenorphine and other phenolic analgesics, to increase the oral availability of the respective analgesic, and to reduce or eliminate pain.

### BACKGROUND OF THE INVENTION

Inadequate pain relief continues to represent a major problem for both patients and healthcare professionals. Optimal pharmacologic management of pain requires selection of the appropriate analgesic drug that achieves rapid efficacy with minimal side effects.

Mild analgesics are readily available, both over the counter (OTC) and by prescription. These include the non-steroidal anti inflammatory drugs (NSAIDs) such as aspirin and ibuprofen, which are well established for the treatment of mild pain. However, while offering effective pain relief they also have side effects such as gastric ulceration and potential for hemorrhage. The other widely used drug for the treatment of mild pain is acetaminophen (paracetamol) but this, in excessive doses, can lead to liver toxicity.

Treatment of more severe pain with opioid analgesics such as oxymorphone may also have their limitations. Unwanted effects can include sedation, respiratory depression, chronic constipation and abuse liability. Many of the stronger opioid analgesics possess a phenolic or hydroxylic function. Such drugs include butorphanol, buprenorphine, codeine, dezocine, dihydrocodeine, hydromorphone, levorphanol, meptazinol, morphine, nalbuphine, oxycodone, oxymorphone, and pentazocine. As a consequence of the presence of either a phenolic or hydroxylic function, many of these compounds are subject to extensive metabolism during the initial passage through the liver after oral dosing, limiting the amount of unchanged drug which can reach the systemic circulation. This high first pass effect results in poor oral bioavailability. For example, meptazinol, oxymorphone and buprenorphine all have oral bioavailabilities less than 10%. A direct consequence of such low bioavailability is considerable variability in attained blood levels within and between subjects. For example, with meptazinol, the range of observed oral bioavailabilities extends from 2-20% (Norbury et al., (1983) Eur. J Clin Pharmacol 25, 77-80). This inevitably results in a variable analgesic response requiring subjects to be individually titrated to achieve adequate pain relief. Dose titration can be tedious and time consuming and make effective treatment of subjects extremely difficult. In any event, the treatment of moderate to severe pain demands urgent relief and subjects may not be prepared to tolerate a protracted period of dose titration. This inevitably leads to compliance issues among subjects.

Several different approaches have been examined to overcome the problems of poor oral bioavaibilities of opioid analgesics, including sublingual or transdermal drug delivery of buprenorphine. For meptazinol, a suppository formulation was developed in an attempt to effect absorption from the superior hemorrhoidal veins, with the goal of avoiding the hepatic portal transfer to the liver. With oxymorphone, no oral formulation was available until 2006, despite the drug being originally introduced to the U.S. market in 1959. Presently available oral formulations of oxymorphone now include a sustained release preparation, but still have a poor oral bioavailability of 10%. This poor oral bioavailability is associated with considerable variation in achieved plasma drug levels.

Thus, despite their pharmacological merits, the use of phenolic analgesics can be compromised by inadequate oral bioavailability. The merits and pharmacokinetic shortcomings of three representative phenolic opioid analgesics—meptazinol, oxymorphone and buprenorphine are discussed in more detail below.

Meptazinol is a mixed agonist-antagonist analgesic with specificity for the μ₁ opioid receptor and displays both opioid (Spiegel and Pasternak (1984). J Pharmacol Exp Ther 228, 414B) and cholinergic properties (Ennis et al. (1986). J Pharm Pharmacol 38, 24-27). As such, it is capable of relieving moderate to moderately severe pain (Siegel et al. (1989). J Clin Pharmacol 29, 1017-1025). Meptazinol exists in two enantiomeric forms, and is used as its racemate. The chemical structure of meptazinol is given below.

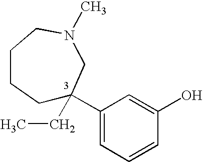

### Meptazinol-3-(3-Ethyl-1-methyl-azepan-3-yl)-phenol

The preparation of a meptazinol hydrobromide salt is taught in U.S. Pat. No. 3,729,465. Preparation of the free base form of meptazinol is taught in U.S. Pat. No. 4,197,241, both of which are incorporated by reference herein in their entireties.

Meptazinol is a potent inhibitor of acetyl choline esterase, and the consequential cholinergic properties are thought to contribute to its anti-nociceptive effects (Bill et al. (1983). Br J Pharmacol 79, 191-199). Additionally, this activity may counter the typical side effects associated with the more traditional opioid therapeutics (Li et al. (2005). Acta Pharmacol Sin 26, 334-338). Meptazinol has also been shown to have a negligible clinical dependency liability from both formal clinical investigation and the lack of reported instances of street use/abuse (Johnson and Jasinski (1987). Clin. Pharm. Ther. 41, 426-33). This negligible clinical dependency distinguishes meptazinol from other opioid analgesics such as fentanyl (Duragesic®), pentazocine, oxycodone (Oxycontin®, Percocet®), and morphine, which are all classified as “Controlled Drugs” and, consequently, have prescription/dispensation restrictions.

Meptazinol has many other clinical advantages over the more conventional opioid analgesics, including lower respiratory depression (Verborgh and Camu (1990). Eur. J. Clin Pharmacol 38, 437-42), minimal sedation (Bradley and Nicholson (1987). Eur. J. Clin Pharmacol 32, 135-139), and lack of a constipating effect (Price and Latham (1982). Curr Ther Res 31, 807-812).

However, despite these clinical advantages, use of meptazinol has been restricted by the major disadvantage of its low oral bioavailability, with reported mean bioavailability values lying between 4-9% (Norbury et al. (1983) Eur J Clin Pharmacol 25, 77-80). The low bioavailability is due to extensive conjugation of meptazinol's metabolically vulnerable phenolic function with glucuronic acid (Franklin (1988). Xenobiotica 18, 105-112). This process can remove up to 98% of an oral dose of meptazinol as it passes through the liver (i.e., first pass metabolism). Such a high first pass elimination of the drug inevitably leads to large inter- and intra-subject variability of plasma drug concentrations and consequent variability in analgesic response. For example, in one report, oral bioavailability varied from 1.89% to 18.5%, almost a ten-fold range (Norbury et al. (1983). Eur. J. Clin. Pharm. 25, 77-80).

Strategies to avoid first pass metabolism of meptazinol have had limited success. For example, a rectal formulation of the drug was tested, and while partially avoiding the first pass effect (bioavailability was increased to some 15-20%), such a route of administration proved to be practically and aesthetically unacceptable (Murray et al. (1989). Eur. J. Clin Pharmacol 36, 279-282). Benzoyl esters and coumarin have been described as possible prodrug moieties for use with meptazinol (Lu et al. (2005) Biorg Med Chem Lett 15, 2607-2690; and Xie et al. (2005). Bioorg. Med. Chem Lett 15, 4953-4956). However, the use of exogenous xenobiotic conjugating prodrug moieties such as coumarin may potentially present additional toxicity issues when cleaved from the meptazinol molecule.

Oxymorphone (Opana®, Numorphan®, Numorphone®) or 14-hydroxydihydromorphinone, is a semi-synthetic μ-opioid agonist analgesic, first developed in Germany around 1914, patented in the USA by Endo Pharmaceuticals in 1955 and introduced to the United States market in January 1959. The preparation of oxymorphone is taught in U.S. Pat. No. 2,806,033. Oxymorphone is approximately 6-8 times more potent than morphine to which it is chemically related (Beaver et al. (1977). J. Clin. Pharmacol. 17, 186-198). Oxymorphone's structure is given below.

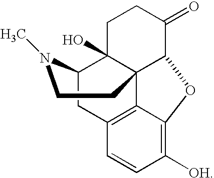

Oxymorphone has a greater affinity than morphine for 1′-opioid receptors, as well as for δ (delta) receptors. The latter activity is believed to potentiate the analgesic effects on the former, while also reducing the risk of tolerance (Chamberlin et al. (2007). Annals of Pharmacotherapy 41, 144-152). Oxymorphone has little affinity for the κ (kappa) receptor (ten fold less than μ or δ) which may explain the drug's decreased sedative properties (Sinatra and Harrison (1989). Clin Pharm. 8, 541-544).

Despite these pharmacological advantages, oxymorphone displays poor pharmacokinetics. The absolute oral bioavailability of the drug is only 10% (Sloan et al. (2005). Supp Care Cancer 13, 57-65), and as a consequence, there is much variability in the attained plasma concentrations and potentially, subject response. This variability is reflected in Cₘₐₓ values, which after a single 5 mg dose, is associated with a >50% (relative standard deviation (RSD). Further, after multiple dosing, the RSD is only reduced to ˜36% (Opana® FDA labeling). Total exposures, reflected in AUC values, are similarly variable. There is also an undesirable food effect on the pharmacokinetics that increases Cₘₐₓ values by up 38% (Opana® FDA labeling). The inherently low oral bioavailability of oxymorphone is the result of rapid and extensive first pass conjugation of the free phenolic function in the molecule (Adams and Ahdieh (2005). Drugs R & D 6, 91-99). The half life of the drug is relatively short (˜7 h) as a consequence of this efficient metabolic clearance, and therefore, necessitates frequent dosing (Adams and Ahdieh (2005). Drugs R & D 6, 91-99).

A probable consequence of the pharmacokinetic inadequacies of oxymorphone is the lack, until recently, of a commercial oral formulation of oxymorphone. However, in 2006, Endo Pharmaceuticals introduced both extended and immediate release oxymorphone oral formulations, called Opana ER® and Opana®, respectively. The latter can be used for so-called rescue therapy. However, neither of the products improved the absolute oral bioavailability of oxymorphone. In addition, variability in plasma drug levels was still observed. Following multiple dosing with 5 mg Opana® ER, maximum plasma concentrations showed a relative standard deviation (RSD) of some 79% (Opana® ER, FDA labeling). Similarly, total exposure expressed as AUC was associated with an RSD of 69% (Opana ER®, FDA labeling).

Historically, various prodrugs of oxymorphone have been proposed including substituted benzoate esters (U.S. Pat. No. 4,668,685).

Buprenorphine is a mixed agonist antagonist opioid analgesic (shown below).

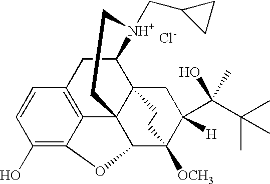

### Buprenorphine Hydrochloride

### [(5α,7α(S)]-17-(Cyclopropylmethyl)-α-(1,1-dimethylethyl)-4,5-epoxy-18,19-dihydro-3-hydroxy-6-methoxy-α-methyl-6,14-ethenomorphinan-7-methanol hydrochloride)

Buprenorphine is not only used clinically as an analgesic, but also as substitution therapy for opioid dependence. The pharmacology of buprenorphine is unique. It acts as a partial agonist at the mu (μ) opioid receptor and also binds to the kappa (κ) receptor (Greenwald et al., Neuropsychopharmacology (2003). 28, 2000-2009). It also interacts with the opioid receptor-like (ORL1) receptor (Yamamoto et al., J Pharmacol Exp Ther. (2006). 318, 206-213). Interactions at the μ receptor produce clinical effects similar to methadone, including analgesia, sedation, euphoria and respiratory depression (Elkader and Sproule (2005). Pharmacokinet 44, 661-680). However as only a partial agonist, buprenorphine has maximal opioid effects lower than the full agonist providing a wider safety margin. Effects at the κ receptor largely result in modest analgesia and some sedation. Presently, there is no conventional oral tablet formulation of buprenophine due to the extremely low oral bioavailability of the drug (less than 10%).

The sublingual preparation Buprenex® was purposefully designed in an attempt to reduce the extent of first pass metabolism by facilitating buccal absorption of the drug, even though, inevitably, a proportion is still swallowed and absorbed through the gut. Nevertheless, this formulation improved the bioavailability of buprenorphine to 30% (Mendleson et al. (1997). J Clin Pharmacol. 37, 31-37). The principal problem with a sublingual formulation is the variability of drug levels in blood. A significant correlation was found between the time taken for the tablet to disintegrate and the peak buprenorphine plasma concentration (Nath et al. (1999). J. Clin. Pharmacol. 39, 619-623). Thus, sublingual administration neither offers a convenient means of drug administration nor a route associated with consistent drug response.

The recently introduced transdermal patches of buprenorphine—Transtec® (3-day patch) and Butrans® (7-day patch) represent alternative means of avoiding first pass metabolism of the drug in the gut wall and liver. This potentially offers an improvement over sublingual dosage by ensuring complete avoidance of absorption though the gut. However, transdermal drug delivery is frequently associated with variations in the rate and extent of absorption depending on the site used for skin application. Furthermore, local skin irritation, specifically erythema and pruritus, typically associated with this route of delivery, has been reported to have an incidence of some 26.6% (erythema) & 23.2% (pruritus) of patients treated with Transtec® patches. (Evans and Easthope (2003). Drugs 63, 1999-2010). Finally, transdermal patches have been historically associated with issues of patch adherence particularly following bathing, showering or swimming. Patches designed to be retained on the skin for extended periods, such as the three (Transtec®) and seven (Butrans®) day buprenorphine transdermal products, are potentially more likely to suffer from such problems.

Because of the unpredictable nature of the plasma drug concentrations after oral administration of phenolic analgesics such as those described above, and a patient's demand for immediate relief from moderate to severe pain, a patient may be unwilling to continue treatment until an optimal dosage is discovered. This frustration in attaining optimal dosage levels for each individual patient can lead to compliance problems. The compliance issue may be exacerbated by the need for frequent oral administration which may be several times per day as a result of rapid clearance.

Due to these disadvantages, the current oral formulations of meptazinol, oxymorphone as well as the currently available formulations of buprenorphine are not ideal for pain relief. Thus, there is clearly an important need for improved oral formulations of these and other phenolic analgesics, in order to increase the respective analgesic's oral bioavailability, as well as to deliver a pharmacologically effective amount of the drug for the treatment of pain and other analgesic benefits. The present invention addresses this and other needs.

### SUMMARY OF THE INVENTION

In one embodiment, the present invention is directed to a compound of Formula I:

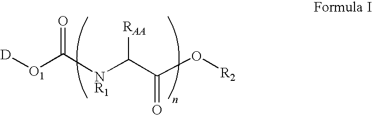

or a pharmaceutically acceptable salt thereof,

wherein,

D is a phenolic analgesic having a low bioavailability,

R₁ and R₂ are independently selected from hydrogen, unsubstituted alkyl, substituted alkyl, cycloalkyl or substituted cycloalkyl group,

R_{AA} is selected from a natural or non-natural amino acid side chain;

O₁ is an oxygen atom present in the unbound form of the opioid analgesic; and

n is an integer from 1 to 9 and

each occurrence of R₁ and R_{AA} can be the same or different.

In some embodiments, n is 1, 2, 3, 4 or 5.

In a preferred embodiment, the prodrug moiety of the compound of Formula I has one, two or three amino acids (i.e., n=1, 2 or 3), while R₂ is H.

In one embodiment, the phenolic analgesic (D) is selected from butorphanol, buprenorphine, codeine, dezocine, dihydrocodeine, hydromorphone, levorphanol, meptazinol, morphine, nalbuphine, oxycodone, oxymorphone, and pentazocine.

In a further embodiment the phenolic narcotic may be a poorly bioavailable opioid antagonist such as naloxone.

In some embodiments, the oral bioavailability of the phenolic analgesic D provided by the compound of Formula I is at least twice the oral bioavailability of the phenolic analgesic D, when administered alone.

In another embodiment, the present invention is directed to a pharmaceutical composition comprising one or more of the opioid prodrugs of the present invention, and one or more pharmaceutically acceptable excipients.

In yet another embodiment, a method of reducing or eliminating pain is provided. The method comprises administering, to a subject in need thereof, an effective amount of the opioid prodrug of the present invention, or a pharmaceutical composition of the present invention.

In a further embodiment, the type of pain which can be treated with the opioid prodrugs of the present invention includes neuropathic pain and nociceptive pain. Other specific types of pain which can be treated with the opioid prodrugs of the present invention include, but are not limited to, acute pain, chronic pain, post-operative pain, pain due to neuralgia (e.g., post herpetic neuralgia or trigeminal neuralgia), pain due to diabetic neuropathy, dental pain, pain associated with arthritis or osteoarthritis, and pain associated with cancer or its treatment.

In one embodiment, the present invention is directed to a method for increasing the oral bioavailability of a phenolic analgesic. The method comprises administering, to a subject in need thereof, an effective amount of the phenolic analgesic carbamate prodrug of the present invention, or a composition of the present invention.

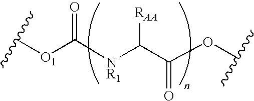

In one embodiment, the moiety of the present invention is selected from valine carbamate, L-met carbamate, 2-amino-butyric acid carbamate, ala carbamate, phe carbamate, ile carbamate, 2-amino acetic acid carbamate, leu carbamate, ala-ala carbamate, val-val carbamate, tyr-gly carbamate, val-tyr carbamate, tyr-val carbamate and val-gly carbamate.

In one embodiment, a method is provided for reducing inter- or intra-subject variability of a phenolic analgesic's plasma levels. The method comprises administering to a subject, or group of subjects, in need thereof, an effective amount of the phenolic analgesic carbamate prodrug of the present invention, or a composition of the present invention.

In a further embodiment, the methods, compounds and compositions of the present invention utilize conjugates of a phenolic analgesic comprising from one to four amino acids, i.e., n is 1, 2, 3 or 4. In yet a further aspect, n is either 1, 2 or 3 and R₂ is H.

In one embodiment, the compounds, compositions and methods of the present invention utilize amino acid and small peptide conjugates of butorphanol, buprenorphine, codeine, dezocine, dihydrocodeine, hydromorphone, levorphanol, meptazinol, morphine, nalbuphine, naloxone, oxycodone, oxymorphone, and pentazocine.

Thus, the present invention relates to natural and/or non-natural amino acids and short-chain peptide prodrugs of phenolic analgesics, for example meptazinol, oxymorphone and buprenorphine, which temporarily protect these analgesics from elimination during, for example, first pass metabolism and deliver a pharmacologically effective amount of the drug for the reduction or elimination of pain. The prodrugs of the present invention provide a viable means for increasing the bioavailability of a phenolic analgesic which has a low bioavailability when administered alone. By reducing the amount of phenolic analgesic that is eliminated during first pass metabolism after oral dosing, the prodrugs of the present invention provide reduced intra- and inter-subject variability in plasma concentrations and, thus, provide for improved analgesic efficacy and better patient compliance.

These and other embodiments of the invention are disclosed or are apparent from and encompassed by the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 shows the plasma concentration of meptazinol in dogs after dosing orally with either meptazinol itself or meptazinol valine carbamate;

FIG. 2 shows the plasma concentration of oxymorphone in dogs after dosing orally with either oxymorphone itself or oxymorphone valine carbamate; and

FIG. 3 shows the plasma concentration of buprenorphine in dogs after dosing orally with either buprenorphine itself or buprenorphine valine carbamate.

### DETAILED DESCRIPTION

### Definitions

As used herein:

The term “peptide” refers to an amino acid chain consisting of 2 to 9 amino acids, unless otherwise specified. In preferred embodiments, the peptide used in the present invention is 2 or 3 amino acids in length.

The term “amino acid” refers both to naturally occurring and non-naturally occurring amino acids, and carbamate derivatives thereof.

A “natural amino acid” is one of the twenty amino acids used for protein biosynthesis as well as other amino acids which can be incorporated into proteins during translation (such as pyrrolysine and selenocysteine). A natural amino acid generally has the formula

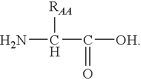

R_{AA} is referred to as the amino acid side chain, or in the case of a natural amino acid, as the natural amino acid side chain. The natural amino acids include glycine, alanine, valine, leucine, isoleucine, aspartic acid, glutamic acid, serine, threonine, glutamine, asparagine, arginine, lysine, proline, phenylalanine, tyrosine, tryptophan, cysteine, methionine and histidine.

Examples of natural amino acid sidechains include hydrogen (glycine), methyl (alanine), isopropyl (valine), sec-butyl (isoleucine), —CH₂CH(CH₃)₂ (leucine), benzyl (phenylalanine), p-hydroxybenzyl (tyrosine), —CH₂OH (serine), —CH(OH)CH₃ (threonine), —CH₂-3-indoyl (tryptophan), —CH₂COOH (aspartic acid), —CH₂CH₂COOH (glutamic acid), —CH₂C(O)NH₂ (asparagine), —CH₂CH₂C(O)NH₂ (glutamine), —CH₂SH, (cysteine), —CH₂CH₂SCH₃ (methionine), —(CH₂)₄NH₂ (lysine), —(CH₂)₃NHC(═NH)NH₂ (arginine) and —CH₂-3-imidazoyl (histidine).

A “non-natural amino acid” is an organic compound that is not among those encoded by the standard genetic code or incorporated into proteins during translation. Non-natural amino acids, thus, include amino acids or analogs of amino acids other than the 20 naturally-occurring amino acids and include, but are not limited to, the D-isostereomers of amino acids. Examples of non-natural amino acids include, but are not limited to: citrulline, hydroxyproline, homoarginine, homoproline, ornithine, 4-amino-phenylalanine, norleucine, cyclohexylalanine, α-aminoisobutyric acid, N-methyl-alanine, N-methyl-glycine, N-methyl-glutamic acid, tert-butylglycine, α-aminobutyric acid, tert-butylalanine, α-aminoisobutyric acid, 2-aminoisobutyric acid 2-aminoindane-2-carboxylic acid, lanthionine, homocitrulline, selenomethionine, dehydroalanine, γ-amino butyric acid, and derivatives thereof wherein the amine nitrogen has been mono- or di-alkylated.

The term “amino” refers to a —NH₂ group;

The term “alkyl,” as a group, refers to a straight or branched hydrocarbon chain containing the specified number of carbon atoms. When the term “alkyl” is used without reference to a number of carbon atoms, it is to be understood to refer to a C₁-C₁₀ alkyl. For example, C₁₋₁₀ alkyl means a straight or branched alkyl containing at least 1, and at most 10, carbon atoms. Examples of “alkyl” as used herein include, but are not limited to, methyl, ethyl, n-propyl, n-butyl, n-pentyl, isobutyl, isopropyl, t-butyl, hexyl, heptyl, octyl, nonyl and decyl.

The term “substituted alkyl” as used herein denotes alkyl radicals wherein at least one hydrogen is replaced by one more substituents such as, but not limited to, hydroxy, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition pertains whether the term is applied to a substituent itself or to a substituent of a substituent.

The term “heterocycle” refers to a stable 3- to 15-membered ring radical which consists of carbon atoms and from one to five heteroatoms selected from nitrogen, phosphorus, oxygen and sulphur.

The term “cycloalkyl” group as used herein refers to a non-aromatic monocyclic hydrocarbon ring of 3 to 8 carbon atoms such as, for example, cyclopropyl, cyclobutyl, cyclopentyl, cyclohexyl or cycloheptyl.

The term “cycloalkyl” group as used herein refers to a non-aromatic monocyclic hydrocarbon ring of 3 to 8 carbon atoms such as, for example, cyclopropyl, cyclobutyl, cyclopentyl, cyclohexyl or cycloheptyl.

The term “substituted cycloalkyl” as used herein denotes a cycloalkyl group further bearing one or more substituents as set forth herein.

The terms “keto” and “oxo” are synonymous and refer to the group ═O;

The term “carbonyl” refers to a group —C(═O);

The term “carboxyl” refers to a group —CO₂H and consists of a carbonyl and a hydroxyl group (More specifically, C(═O)OH);

The terms “carbamate group,” and “carbamate,” concerns the group

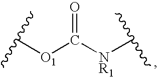

wherein the —O₁— is present in the unbound form of the opioid analgesic. Prodrug moieties described herein may be referred to based on their amino acid or peptide and the carbamate linkage. The amino acid or peptide in such a reference should be assumed to be bound via an amino terminus on the amino acid or peptide to the carbonyl linker and the opioid analgesic, unless otherwise specified.

For example, val carbamate (valine carbamate) would have the formula

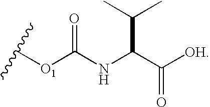

For a peptide, such as tyr-val carbamate, it should be assumed unless otherwise specified that the leftmost amino acid in the peptide is at the amino terminus of the peptide, and is bound via the carbonyl linker to the opioid analgesic to form the carbamate prodrug.

The term “carrier” refers to a diluent, excipient, and/or vehicle with which an active compound is administered. The pharmaceutical compositions of the invention may contain one or a combination of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil and sesame oil. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18^{th} Edition.

The phrase “pharmaceutically acceptable” refers to molecular entities and compositions that are generally regarded as safe. In particular, pharmaceutically acceptable carriers used in the practice of this invention are physiologically tolerable and do not typically produce an allergic or similar untoward reaction (for example, gastric upset, dizziness) when administered to a subject. Preferably, as used herein, the term “pharmaceutically acceptable” means approved by a regulatory agency of the appropriate governmental agency or listed in the U.S. Pharmacopoeia or other generally recognized pharmacopoeia for use in animals, and more particularly in humans.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The term “treating” includes: (1) preventing or delaying the appearance of clinical symptoms of the state, disorder or condition developing in an animal that may be afflicted with or predisposed to the state, disorder or condition but does not yet experience or display clinical or subclinical symptoms of the state, disorder or condition; (2) inhibiting the state, disorder or condition (i.e., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a subject to be treated is either statistically significant or at least perceptible to the subject or to the physician.

“Effective amount” means an amount of an opioid prodrug used in the present invention sufficient to result in the desired therapeutic response. The therapeutic response can be any response that a user (e.g., a clinician) will recognize as an effective response to the therapy. The therapeutic response will generally be analgesic response affording pain relief. It is further within the skill of one of ordinary skill in the art to determine an appropriate treatment duration, appropriate doses, and any potential combination treatments, based upon an evaluation of therapeutic response.

The term “subject” includes humans and other mammals, such as domestic animals (e.g., dogs and cats).

The term “salts” can include acid addition salts or addition salts of free bases. Suitable pharmaceutically acceptable salts (for example, of the carboxyl terminus of the amino acid or peptide) include, but are not limited to, metal salts such as sodium potassium and cesium salts; alkaline earth metal salts such as calcium and magnesium salts; organic amine salts such as triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Pharmaceutically acceptable salts (of basic nitrogen centers) include, but are not limited to inorganic acid salts such as the hydrochloride, hydrobromide, sulfate, phosphate; organic acid salts such as trifluoroacetate and maleate salts; sulfonates such as methanesulfonate, ethanesulfonate, benzenesulfonate, p-toluenesulfonate, camphor sulfonate and naphthalenesulfonate; and amino acid salts such as arginate, gluconate, galacturonate, alaninate, asparginate and glutamate salts (see, for example, Berge, et al. “Pharmaceutical Salts,” J. Pharma. Sci. 1977; 66:1).

The term “active ingredient,” unless specifically indicated, is to be understood as referring to the phenolic analgesic portion of the prodrug, described herein.

The term “bioavailability” generally means the rate and/or extent to which the active ingredient is absorbed from a drug product and becomes systemically available and hence available at the site of action. See Code of Federal Regulations, Title 21, Part 320.1 (2003 ed.). For oral dosage forms, bioavailability relates to the processes by which the active ingredient is released from the oral dosage form and becomes systemically available and hence available at the site of action. Bioavailability data for a particular formulation provides an estimate of the fraction of the administered dose that is absorbed into the systemic circulation. Thus, the term “oral bioavailability” refers to the fraction of a dose of a drug given orally that reaches the systemic circulation after a single administration to a subject. A preferred method for determining the oral bioavailability is by dividing the AUC of the drug given orally by the AUC of the same dose given intravenously to the same subject, and expressing the ratio as a percent. Other methods for calculating oral bioavailability will be familiar to those skilled in the art, and are described in greater detail in Shargel and Yu, Applied Biopharmaceutics and Pharmacokinetics, 4th Edition, 1999, Appleton & Lange, Stamford, Conn., incorporated herein by reference in its entirety.

The term “increase in oral bioavailability” refers to the increase in the bioavailability of the drug when orally administered as a prodrug of the present invention (either a prodrug compound or composition), as compared to the bioavailability when the drug is orally administered alone. The increase in oral bioavailability can be from 5% to 20,000%, preferably from 200% to 20,000%, more preferably from 500% to 20,000%, and most preferably from 1000% to 20,000%.

The term “low oral bioavailability,” refers to an oral bioavailability wherein the fraction of a dose of the parent drug given orally that is absorbed into the plasma unchanged after a single administration to a subject is 25% or less, preferably 15% or less, and most preferably 10% or less. Without wishing to be bound by any particular theory, it is believed that the low oral bioavailability of the respective phenolic analgesics described herein, is the result of the conjugation of a phenolic oxygen in the phenolic analgesic to glucuronic acid, during first pass metabolism. However, other mechanisms may be responsible for the decrease in oral bioavailability and are also contemplated by the present invention.

Compounds of the Invention

The present invention is directed to amino acid and peptide prodrugs that increase the oral bioavailability of a phenolic analgesic, as compared to oral administration of the phenolic analgesic alone.

Without wishing to be bound to any particular theory, it is believed that absorption of the phenolic analgesic carbamate prodrugs presented herein selectively exploit the inherent di- and tri-peptide transporter Pept1 within the digestive tract to facilitate their absorption.

Once absorbed, the carbamate prodrugs provide sufficient temporary protection against the gut wall and hepatic conjugation of the analgesic's phenolic functionality with glucuronic acid to ensure that a significantly larger amount of the respective phenolic analgesic reaches the systemic circulation. It is believed that the phenolic analgesic is released from the amino acid or peptide prodrug by hepatic and extrahepatic hydrolases that are, in part, present in plasma.

The use of the prodrugs of the present invention will provide greater consistency in analgesic response as the result of higher oral bioavailability offering a significant reduction in the extent of inter- and intra-subject variability in plasma and CNS concentrations and, hence, significantly less fluctuation in pain relief. Thus, patient/subject compliance is likely to be further improved as the result of this greater predictability of analgesic response.

The amino acid and peptide carbamate prodrugs of the chiral phenolic analgesics disclosed in the present invention can be either single diastereoisomers or mixtures of diastereoisomers.

In one embodiment, the present invention is directed to a phenolic analgesic carbamate prodrug of Formula I:

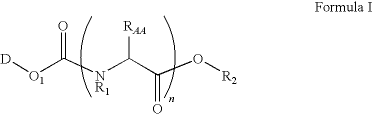

or a pharmaceutically acceptable salt thereof,

wherein,

D is a phenolic analgesic having a low bioavailability,

R₁ and R₂ are independently selected from hydrogen, unsubstituted alkyl, substituted alkyl, cycloalkyl or substituted cycloalkyl group,

R_{AA} is selected from a natural or non-natural amino acid side chain;

O₁ is an oxygen atom present in the unbound form of the opioid analgesic; and

n is an integer from 1 to 9 and

each occurrence of R₁ and R_{AA} can be the same or different.

In some embodiments, n is 1, 2, 3, 4 or 5.

In a preferred embodiment, the prodrug moiety of the compound of Formula I has one, two or three amino acids (i.e., n=1, 2 or 3), while R₂ is H.

In one embodiment, the phenolic analgesic (D) is selected from butorphanol, buprenorphine, codeine, dezocine, dihydrocodeine, hydromorphone, levorphanol, meptazinol, morphine, nalbuphine, oxycodone, oxymorphone, and pentazocine. In another embodiment, the phenolic narcotic is naloxone.

In various embodiments, the oral bioavailability of the phenolic analgesic D provided by the compound of Formula I is at least twice the oral bioavailability of the phenolic analgesic D, when administered alone.

The 20 naturally occurring amino acids used for protein biosynthesis, as well as their abbreviations, are given in Table 1 below.

TABLE 1
Natural Amino acids (used for protein
biosynthesis) and Their Abbreviations

| Amino acid | 3 letter code | 1-letter code |
|---|---|---|
| Alanine | ALA | A |
| Cysteine | CYS | C |
| Aspartic Acid | ASP | D |
| Glutamic Acid | GLU | E |
| Phenylalanine | PHE | F |
| Glycine | GLY | G |
| Histidine | HIS | H |
| Isoleucine | ILE | I |
| Lysine | LYS | K |
| Leucine | LEU | L |
| Methionine | MET | M |
| Asparagine | ASN | N |
| Proline | PRO | P |
| Glutamine | GLN | Q |
| Arginine | ARG | R |
| Serine | SER | S |
| Threonine | THR | T |
| Valine | VAL | V |
| Tryptophan | TRP | W |
| Tyrosine | TYR | Y |

The amino acids employed in the prodrugs for use with the present invention are preferably in the L configuration (i.e., have a negative optical rotation). The present invention also contemplates prodrugs of the invention comprised of amino acids in the D configuration, or mixtures of amino acids in the D and L configurations.

Meptazinol Prodrugs of the Present Invention

In one embodiment of the present invention, the prodrugs are novel amino acid and peptide prodrugs of meptazinol. Preferably, these prodrugs comprise meptazinol attached to a single amino acid or a short peptide through a carbamate linkage, wherein the carbamate is attached to the metabolically vulnerable phenolic function of meptazinol. This preferred modification to meptazinol radically improves the otherwise very poor oral bioavailability (<10%) of meptazinol. The low oral bioavailability of meptazinol when administered alone—and its inherently variable bioavailability—has resulted in the need for tedious individual subject titration which often results in the abandonment of treatment by the subject. The use of the meptazinol prodrugs of the present invention increases the oral bioavailability of meptazinol by 2 to 10 times (i.e., a 200 to 1000% increase in oral bioavailability).

The novel meptazinol carbamate prodrugs of the present invention include prodrugs of Formula II:

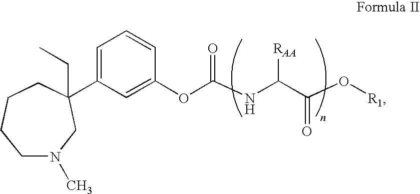

or a pharmaceutically acceptable salt thereof,

wherein,

R₁ is H, an unsubstituted alkyl group, or a substituted alkyl group,

n is an integer from 1 to 9;

R_{AA} is a natural or non-natural amino acid side chain; and each occurrence of R_{AA} can be the same or different;

In one embodiments, n is 1, 2 or 3.

In a preferred embodiment, n is 1, 2 or 3 and R₁ is H.

In another embodiment, n is 1.

In yet another embodiment, n is 2.

In yet another embodiment, n is 1 or 2 and each occurrence of R_{AA} is independently a natural amino acid side chain.

In one embodiment, the oral bioavailability of meptazinol provided by the carbamate prodrug of Formula II is at least twice the oral bioavailability of meptazinol, when administered alone.

Single amino acid meptazinol prodrugs of the present invention include meptazinol-(S)-ile carbamate, meptazinol-(S)-leu carbamate, meptazinol-(S)-asp carbamate, meptazinol-(S)-met carbamate, meptazinol-(S)-his carbamate, meptazinol-(S)-phe carbamate and meptazinol-(S)-ser carbamate.

In a preferred embodiment of the meptazinol prodrugs of Formula (II), at least one occurrence of R_{AA} is the side chain of a non-polar or an aliphatic amino acid. This embodiment includes the single amino acid prodrugs meptazinol valine carbamate, meptazinol isoleucine carbamate, and meptazinol methionine carbamate, the structures of which are represented below. In an especially preferred embodiment, the single amino acid prodrug of Formula (II) is the hydrochloride salt of meptazinol-L-valine carbamate (Common Name: 2-[3-(3-Ethyl-1-methyl-azepin-3-yl)-phenoxycarbonyl amino]-3-methyl-butyric acid hydrochloride).

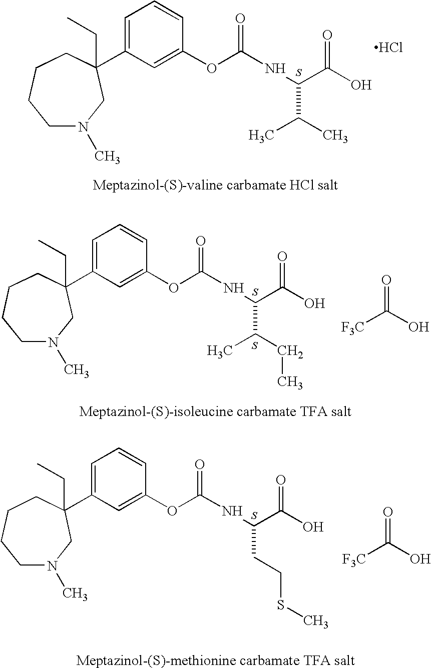

Other single amino acid prodrugs of meptazinol include meptazinol alanine carbamate, meptazinol-2-amino-butyric acid carbamate, meptazinol-L-methionine carbamate, and meptazinol glycyl-2-amino acetic acid carbamate.

Additional embodiments of the meptazinol prodrugs of Formula (II) are dipeptide prodrugs wherein R_{AA} is independently selected from the side chains of non-polar and aliphatic amino acids including valine, glycine and alanine. For example meptazinol-valine-valine carbamate, meptazinol-valine-glycine carbamate, and meptazinol-valine-alanine carbamate are encompassed by the present invention.

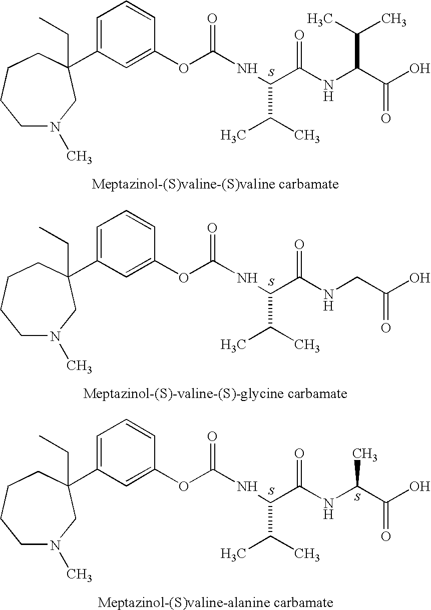

Other dipeptide prodrugs of meptazinol include meptazinol-tyrosine-valine carbamate, meptazinol-tyrosine-glycine-carbamate, and meptazinol-valine-tyrosine carbamate. Alternatively permutations drawn from valine, leucine, isoleucine, alanine and glycine are further embodiments. Yet further embodiments may include permutations drawn from these nonpolar aliphatic amino acids with the nonpolar aromatic amino acids, tryptophan and tyrosine.

The preferred amino acids described above are all in the L configuration, however, the present invention also contemplates prodrugs of Formulae I-XI comprised of amino acids in the D configuration, or mixtures of amino acids in the D and L configurations.

Oxymorphone Prodrugs of the Present Invention

In one embodiment, prodrugs of the present invention are directed to novel oxymorphone prodrugs of Formula III, below.

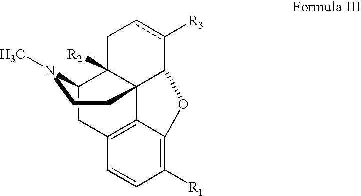

or a pharmaceutically acceptable salt thereof,

wherein,

R₁ and R₂ are selected from

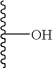

and

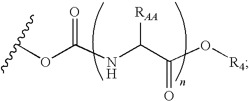

the dashed line in Formula III is absent when R₃ is

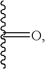

and a bond when R₃ is not

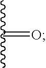

R₃ is selected from

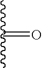

and;

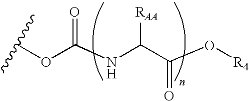

R₄ is independently selected from hydrogen, a substituted alkyl group and an unsubstituted alkyl group;

R_{AA} is a natural or non-natural amino acid side chain, and each occurrence of R_{AA} can be the same or different;

n is an integer selected from 1 to 9 and each occurrence of n can be the same or different;

and at least one of R₁, R₂, and R₃ is

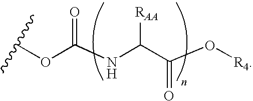

In one embodiments, n is 1, 2 or 3.

In a preferred embodiment, n is 1, 2 or 3 and R₄ is H.

In another embodiment, n is 1.

In yet another embodiment, n is 2.

In yet another embodiment, n is 1 or 2 and each occurrence of R_{AA} is independently a natural amino acid side chain.

In one embodiment, oxymorphone prodrugs of Formulae IV-VII are provided. For Formulae IV-VII, R₄, R_{AA} and n are defined as for Formula III. In one embodiments, n is 1, 2, 3 or 4 and R₄ is H. Each occurrence of n and R_{AA} can be the same, or different.

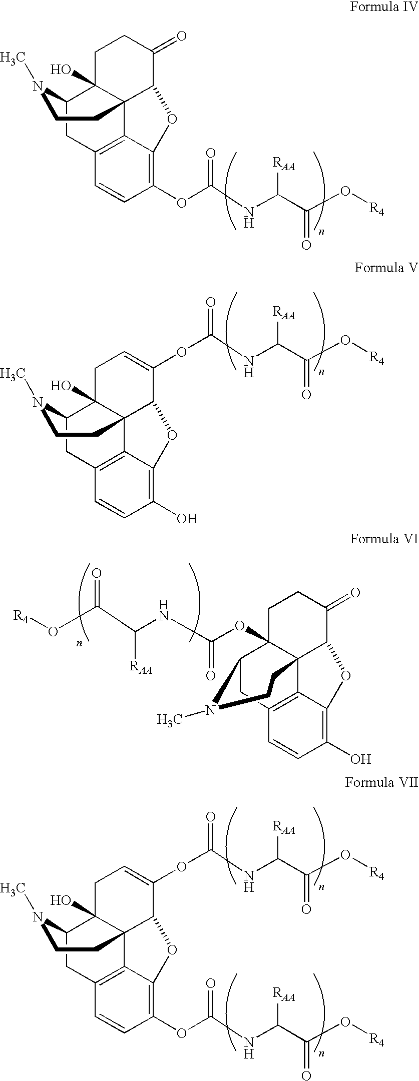

In one embodiment, the oral bioavailability of oxymorphone provided by the compound of any of Formulae III-VII is at least twice the oral bioavailability of oxymorphone, when administered alone.

In some embodiments, the invention is directed to the following oxymorphone carbamate prodrugs—oxymorphone-S-ile carbamate, oxymorphone-S-leu carbamate, oxymorphone-S-asp carbamate, oxymorphone-S-met carbamate, oxymorphone-S-his carbamate, oxymorphone-S-phe carbamate and oxymorphone-S-ser carbamate.

A preferred embodiment of the oxymorphone prodrug of Formula (III) is when the prodrug contains an amino acid side chain of a non-polar or an aliphatic amino acid. This embodiment includes the following prodrugs—oxymorphone valine carbamate, oxymorphone isoleucine carbamate, and oxymorphone methionine carbamate, the structures of which are represented below.

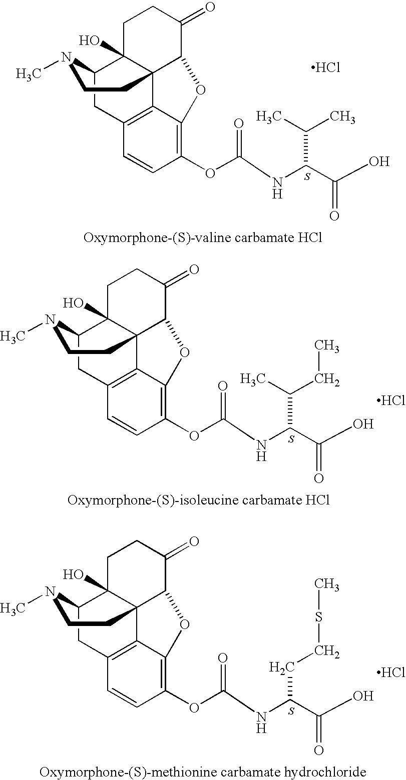

Another preferred embodiment is the single amino acid prodrug of Formula (III) as the hydrochloride salt of oxymorphone valine carbamate (Common Name: (S)-2-[(4,5-Epoxy-14-hydroxy-17-methylmorphinan-6-one-3-yl)-oxycarbonylamino]-3-methylbutanoic acid Hydrochloride).

In a preferred oxymorphone dipeptide carbamate embodiment, the prodrug of the present invention can oxymorphone-valine-valine carbamate, oxymorphone-valine-methionine carbamate, and oxymorphone-valine-isoleucine carbamate. Yet further embodiments may include permutations drawn from a range of aliphatic & aromatic amino acids.

Buprenorphine Prodrugs of the Present Invention

The novel buprenorphine compounds of the present invention include prodrugs of Formula VIII:

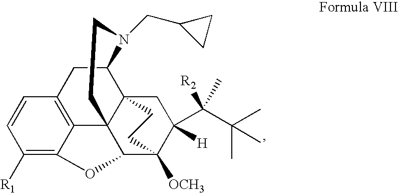

or a pharmaceutically acceptable salt thereof,

wherein,

R₁ and R₂ are selected from

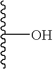

and

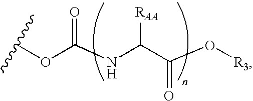

n is an integer from 1 to 9 and each occurrence of n can be the same or different.

R_{AA} is a natural or non-natural amino acid side chain and each occurrence of R_{AA} can be the same or different;

each occurrence of R₃ is selected from H, an unsubstituted alkyl group, or a substituted alkyl group,

and at least one of R₁ and R₂ is

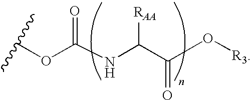

In one embodiments, n is 1, 2 or 3.

In a preferred embodiment, n is 1, 2 or 3 and R₃ is H.

In another embodiment, n is 1.

In yet another embodiment, n is 2.

In yet another embodiment, n is 1 or 2 and each occurrence of R_{AA} is independently a natural amino acid side chain.

In another buprenorphine embodiment, a compound of the present invention is directed to a compounds of any of Formulae IX-XI, shown below. R₃, R_{AA} and n are defined in the same manner as defined for Formula VII. Each occurrence of R_{AA} and n can be the same, or different;

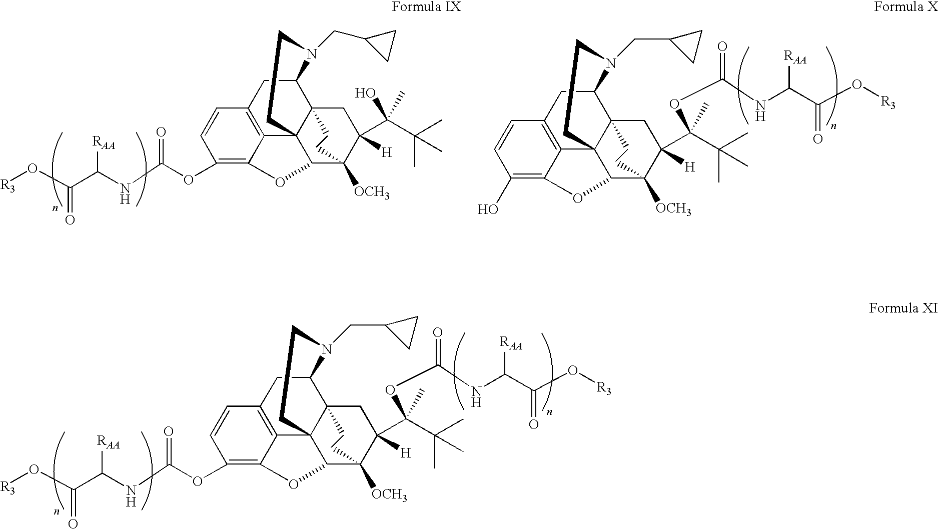

In one embodiment, the oral bioavailability of buprenorphine provided by the compound of any of Formulae VIII-XI is at least twice the oral bioavailability of buprenorphine, when administered alone.

Single amino acid buprenorphine prodrugs of the present invention include buprenorphine-(S)-ile carbamate, buprenorphine-(S)-leu carbamate, buprenorphine-(S)-asp carbamate, buprenorphine-(S)-met carbamate, buprenorphine-(S)-his carbamate, buprenorphine-(S)-phe carbamate and buprenorphine-(S)-ser carbamate.

A preferred embodiment of the prodrugs of Formula (VIII) includes prodrugs that contain an amino acid side chain of a non-polar or an aliphatic amino acid. Accordingly, the buprenorphine carbamate prodrugs in this embodiment include the single amino acid prodrugs buprenorphine valine carbamate, buprenorphine isoleucine carbamate, and buprenorphine leucine carbamate, the structures of which are represented below.

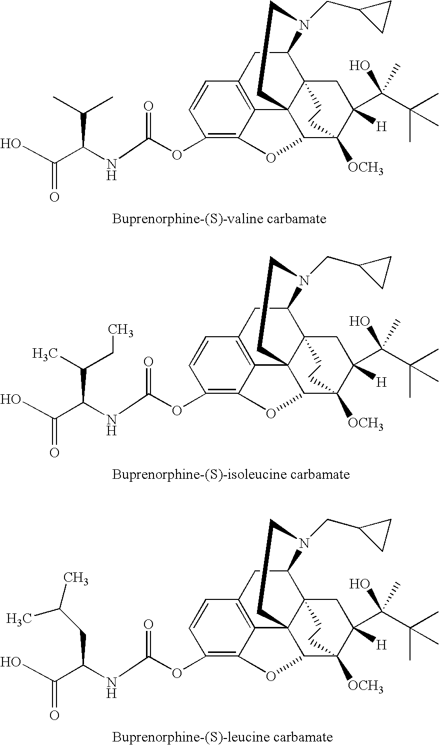

An especially preferred embodiment is the single amino acid prodrug of Formula (IV) as the hydrochloride salt of buprenorphine valine carbamate (Common Name: (S)-2-{[[5α, 7α(S)]-17-(Cyclopropylmethyl)-α-(1,1-dimethylethyl)-4,5-epoxy-18,19-dihydro-6-methoxy-α-methyl-6,14-ethenomorphinan-7-methanol-3-yl]oxycarbonylamino}-3-methylbutyric acid.

In a preferred buprenorphine embodiment, the compounds of the present invention include buprenorphine-valine-valine carbamate, buprenorphine-valine-leucine carbamate, and oxymorphone-valine-isoleucine carbamate. Yet further embodiments may include permutations drawn from a range of aliphatic & aromatic amino acids.

Salts, Solvates, Stereoisomers, Derivatives of the Compounds of the Invention

The methods of the present invention further encompass the use of salts, solvates, stereoisomers of the prodrugs of phenolic analgesics described herein, for example salts of the prodrugs of Formula I, given above. In one embodiment, the invention disclosed herein is meant to encompass all pharmaceutically acceptable salts of meptazinol prodrugs (including those of the carboxyl terminus of the amino acid as well as those of the weakly basic azepine nitrogen).

Typically, a pharmaceutically acceptable salt of a prodrug of a phenolic analgesic used in the practice of the present invention is prepared by reaction of the prodrug of a phenolic analgesic with a desired acid or base as appropriate. The salt may precipitate from solution and be collected by filtration or may be recovered by evaporation of the solvent. For example, an aqueous solution of an acid such as hydrochloric acid may be added to an aqueous suspension of the prodrug of a phenolic analgesic and the resulting mixture evaporated to dryness (lyophilized) to obtain the acid addition salt as a solid. Alternatively, the prodrug of a phenolic analgesic may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The acid addition salts of the prodrugs of a phenolic analgesic may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of the acidic compounds are prepared by contacting the free acid form with a sufficient amount of the desired base to produce the salt in the conventional manner. The free acid form may be regenerated by contacting the salt form with an acid and isolating the free acid.

Compounds useful in the practice of the present invention may have both a basic and an acidic center and may therefore be in the form of zwitterions.

Those skilled in the art of organic chemistry will appreciate that many organic compounds can form complexes, i.e., solvates, with solvents in which they are reacted or from which they are precipitated or crystallized, e.g., hydrates with water. The salts of compounds useful in the present invention may form solvates such as hydrates useful therein. Techniques for the preparation of solvates are well known in the art (see, for example, Brittain. Polymorphism in Pharmaceutical Solids. Marcel Decker, New York, 1999). The compounds useful in the practice of the present invention can have one or more stereogenic centers and, depending on the nature of individual substituents, they can also have geometrical isomers.

Individual isomers of the prodrugs of a phenolic analgesics described herein may be used to practice the present invention. The description or naming of a particular compound in the specification and claims is intended to include both individual enantiomers and mixtures, racemic or otherwise, thereof. Methods for the determination of stereochemistry and the resolution of stereoisomers are well-known in the art.

Pharmaceutical Compositions of the Invention

While it is possible that, for use in the methods of the invention, the prodrug may be administered as the bulk substance, it is preferable to present the active ingredient in a pharmaceutical formulation, e.g., wherein the agent is in admixture with a pharmaceutically acceptable carrier selected with regard to the intended route of administration and standard pharmaceutical practice.

Therefore, in some embodiments, the present invention is directed to a composition comprising a phenolic analgesic carbamate prodrug and a pharmaceutically acceptable excipient. The prodrug can be any prodrug of Formulae I-XI.

The formulations of the invention may be immediate-release dosage forms, i.e., dosage forms that release the prodrug at the site of absorption immediately, or controlled-release dosage forms, i.e., dosage forms that release the prodrug over a predetermined period of time. Controlled release dosage forms may be of any conventional type, e.g., in the form of reservoir or matrix-type diffusion-controlled dosage forms; matrix, encapsulated or enteric-coated dissolution-controlled dosage forms; or osmotic dosage forms. Dosage forms of such types are disclosed, for example, in Remington, The Science and Practice of Pharmacy, 20th Edition, 2000, pp. 858-914. The formulations of the present invention can be administered from one to six times daily, depending on the dosage form and dosage.

Prodrugs of phenolic opioid analgesics which do not result in sustained plasma drugs levels due to continuous generation of the opioid analgesic from a plasma reservoir of prodrug may require formulations that provide a sustained release of the opioid analgesic. For example, formulations that offer gastroretentive or mucoretentive benefits, analogous to those used in metformin products such as Glumetz® or Gluphage XR®, may be employed. The former exploits a drug delivery system known as Gelshield Diffusion™ Technology while the latter uses a so-called Acuform™ delivery system. In both cases, the concept is to retain drug in the stomach, slowing drug passage into the ileum, maximizing the period over which absorption take place and effectively prolonging plasma drug levels. Other drug delivery systems affording delayed progression along the GI tract may also be of value.

In one embodiment, the present invention provides a pharmaceutical composition comprising at least one active pharmaceutical ingredient (i.e., a prodrug of a phenolic analgesic), or a pharmaceutically acceptable derivative (e.g., a salt or solvate) thereof, and a pharmaceutically acceptable carrier. In particular, the invention provides a pharmaceutical composition comprising a therapeutically effective amount of at least one prodrug of the present invention, or a pharmaceutically acceptable derivative thereof, and a pharmaceutically acceptable carrier.

For the methods of the invention, the prodrug employed in the present invention may be used in combination with other therapies and/or active agents. Accordingly, the present invention provides, in a further aspect, a pharmaceutical composition comprising at least one compound useful in the practice of the present invention, or a pharmaceutically acceptable salt or solvate thereof, a second active agent, and, optionally a pharmaceutically acceptable carrier.

For example, the prodrugs of the present invention may be administered to a subject in combination with other active agents used in the management of pain. An active agent to be administered in combination with the prodrugs encompassed by the present invention may include, for example, a drug selected from the group consisting of non-steroidal anti-inflammatory drugs (e.g., ibuprofen), anti-emetic agents (e.g., ondansetron, domerperidone, hyoscine and metoclopramide), unabsorbed or poorly bioavailable opioid antagonists to reduce the risk of drug abuse (e.g., naloxone). In such combination therapies, the prodrugs encompassed by the present invention may be administered prior to, concurrent with, or subsequent to the other therapy and/or active agent.

When combined in the same formulation it will be appreciated that the two compounds must be stable and compatible with each other and the other components of the formulation. When formulated separately they may be provided in any convenient formulation, conveniently in such manner as are known for such compounds in the art.

The prodrugs used herein may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may also be used.

The compounds used in the invention may be milled using known milling procedures such as wet milling to obtain a particle size appropriate for tablet formation and for other formulation types. Finely divided (nanoparticulate) preparations of the compounds may be prepared by processes known in the art, for example see International Patent Application No. WO 02/00196 (SmithKline Beecham).

The compounds and pharmaceutical compositions of the present invention are intended to be administered orally (e.g., as a tablet, sachet, capsule, pastille, pill, boluse, powder, paste, granules, bullets or premix preparation, ovule, elixir, solution, suspension, dispersion, gel, syrup or as an ingestible solution). In addition, compounds may be present as a dry powder for constitution with water or other suitable vehicle before use, optionally with flavoring and coloring agents. Solid and liquid compositions may be prepared according to methods well-known in the art. Such compositions may also contain one or more pharmaceutically acceptable carriers and excipients which may be in solid or liquid form.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions useful herein include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers useful herein include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants useful herein include, but are not limited to, sodium lauryl sulfate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetra-acetic acid (EDTA), thiourea, tocopherol and butyl hydroxyanisole.

The pharmaceutical compositions of the invention may contain from 0.01 to 99% weight per volume of the prodrugs encompassed by the present invention.

Dosages

Appropriate subjects to be treated according to the methods of the invention include any human or animal in need of such treatment. Methods for the diagnosis and clinical evaluation of pain, including the severity of the pain experienced by an animal or human are well known in the art. Thus, it is within the skill of the ordinary practitioner in the art (e.g., a medical doctor or veterinarian) to determine if a subject is in need of treatment for pain. The subject is preferably a mammal, more preferably a human, but can be any animal, including a laboratory animal in the context of a clinical trial or screening or activity experiment employing an animal model. Thus, as can be readily appreciated by one of ordinary skill in the art, the methods and compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, research animals, such as mice, rats, rabbits, goats, sheep, pigs, dogs, cats, etc., avian species, such as chickens, turkeys, songbirds, etc.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. For highly potent agents such as buprenorphine, the daily dose requirement is likely to range from 0.5 to 50 mg, preferably from 1 to 25 mg, and more preferably from 1 mg to 10 mg. For less potent agents such as meptazinol, the daily dose requirement is likely to range from 1 mg to 1600 mg, preferably from 1 mg to 800 mg and more preferably from 1 mg to 400 mg.

The doses referred to herein, unless otherwise indicated, are the amount of phenolic analgesic, in free base form (in mg).

Depending on the severity of pain to be treated, a suitable therapeutically effective and safe dosage, as may readily be determined within the skill of the art, and without undue experimentation, maybe administered to subjects. For oral administration to humans, the daily dosage level of the prodrug may be in single or divided doses. The duration of treatment may be determined by one of ordinary skill in the art, and should reflect the nature of the pain (e.g., a chronic versus an acute condition) and/or the rate and degree of therapeutic response to the treatment.

Where the prodrugs encompassed by the present invention are administered in conjunction with another active agent, the individual components of such combinations may be administered either sequentially or simultaneously in separate or combined pharmaceutical formulations by any convenient route. When administration is sequential, either the prodrugs encompassed by the present invention or the second active agent may be administered first. For example, in the case of a combination therapy with another active agent, the prodrugs encompassed by the present invention may be administered in a sequential manner in a regimen that will provide beneficial effects of the drug combination. When administration is simultaneous, the combination may be administered either in the same or different pharmaceutical compositions. For example, the prodrugs encompassed by the present invention and another active agent may be administered in a substantially simultaneous manner, such as in a single capsule or tablet having a fixed ratio of these agents or in multiple, separate capsules or tablets for each agent.

When the prodrugs encompassed by the present invention are used in combination with another agent active in the methods for treating pain, the dose of each compound may differ from that when the compound is used alone. Appropriate doses will be readily appreciated by those skilled in the art.

### EXAMPLES

The present invention is further illustrated by reference to the following Examples. However, it should be noted that these Examples, like the embodiments described above, are illustrative and are not to be construed as restricting the enabled scope of the invention in any way.

Preparation of the Carbamate Prodrugs of the Invention

Compounds employed in the present invention and derivatives thereof may be prepared by the methods outlined herein. The foregoing examples illustrate the preparation of amino acid prodrug carbamate prodrug conjugates, wherein the selected amino acid is valine. However, these methods may be employed to synthesize any of amino acid prodrugs provided for in Formulae I-XI. Additionally, di- and higher order oligopeptide prodrugs can be prepared using the methods presented in the following examples, combined with synthetic methods that are well known in the art of peptide synthesis. For example, the meptazinol prodrug can be prepared by reacting meptazinol with a di- or higher order oligopeptide, or by reacting meptazinol with single amino acid followed by reacting the single amino acid prodrug with additional amino acids or peptides. Furthermore, the methods illustrated in the following examples can also be used to prepare prodrugs of the present invention wherein the active drug is any phenolic analgesic having low oral bioavailability.

It will be appreciated by those skilled in the art that it may be desirable to use protected derivatives of intermediates used in the preparation of the prodrugs of the present invention. Protection and deprotection of functional groups may be performed by methods known in the art. Hydroxyl or amino groups may be protected with any hydroxyl or amino protecting group (for example, as described in Green and Wuts. Protective Groups in Organic Synthesis. John Wiley and Sons, New York, 1999). The protecting groups may be removed by conventional techniques. For example, acyl groups (RCO where R is an alkyl group and ArCO where Ar is an aryl group) may be removed by hydrolysis under acidic or basic conditions. Arylmethoxycarbonyl groups (e.g., benzyloxycarbonyl) may be cleaved by hydrogenolysis in the presence of a catalyst such as palladium-on-carbon.

The synthesis of the desired prodrug is completed by removing any protecting groups, which are present in the penultimate intermediate using standard techniques. These techniques are well-known to those skilled in the art. The deprotected final product is then purified, as necessary, using standard techniques such as chromatography on silica, HPLC on reverse phase silica and the like, or by recrystallization.

In the following Examples:

Chemicals were purchased primarily from Aldrich Chemical Company, Gillingham, Dorset and Alfa Aesar, Morecambe, Lancashire, U.K. and were used without further purification. Solvents used were anhydrous. Petrol employed was the fraction boiling in the range 40-60° C.

TLC was carried out using aluminum plates pre-coated with silica (Kieselgel 60 F₂₅₄, 0.2 mm, Merck, Darmstadt, Germany). Visualization was by UV light or by dipping in aqueous KMnO₄ and heating. Silica (‘flash’, Kieselgel 60) was used for medium pressure chromatography.

¹H NMR spectra were recorded on a Bruker Avance BVT3200 spectrometer using the residual hydrogen(s) of deuterated solvents as internal standard.

Combustion analyses were performed by Advanced Chemical and Material Analysis, Newcastle University, U.K. using a Carlo-Erba 1108 elemental analyzer.

### Example 1

### Generic Route of Synthesis of Amino Acid Carbamate Conjugates of Opioids

A route to phenolic opioid prodrugs as HCl or TFA salts via amino acid tert-butyl esters (using valine as an example) is provided in Scheme 1, below.

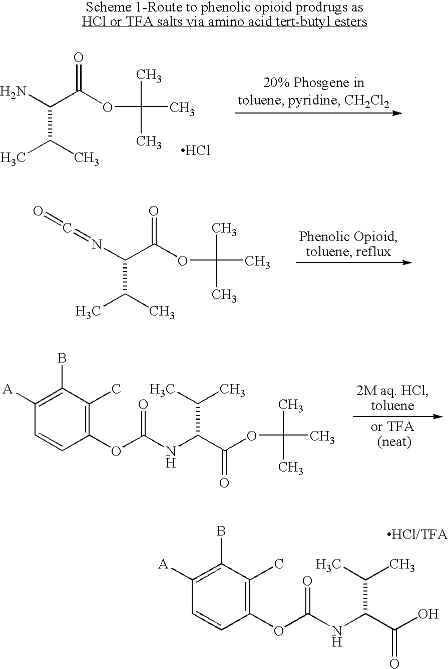

A route to phenolic opioid prodrugs via amino acid benzyl esters is given in Scheme 2, below (using valine as an example).

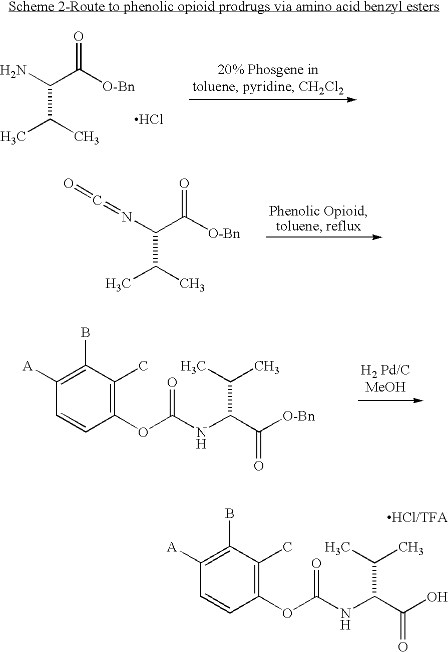

The first route (using tert-butyl esters) is suitable for non-acid sensitive phenolic opiods, whereas the second route (using amino acid benzyl esters) is suitable for those which are acid sensitive but do not contain any reducible functionalities such as double bonds.

### Example 2

### Synthesis of Meptazinol-(S)-Valine Carbamate Trifluoracetate

The synthesis of meptazinol-(S)-valine-carbamate is provided in Scheme 3, below.

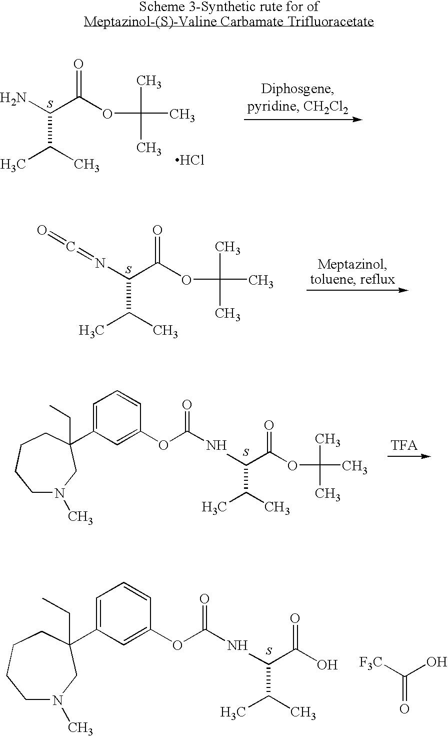

Pyridine (1.56 mL, 19.3 mmol, 1.52 g) was added to a suspension of (s)-valine tert-butyl ester hydrochloride (1.0 g, 4.77 mmol) in anhydrous dichloromethane (30 mL) under nitrogen. The mixture was stirred and cooled in an ice bath, followed by the dropwise addition of diphosgene (0.37 mL, 3.10 mmol, 0.61 g) to the reaction mixture. The reaction mixture was then allowed to warm to room temperature, while stirring was continued for 2 hours. The mixture was diluted with dichloromethane and washed with ice-cold 1M hydrochloric acid and brine. The organic layer was dried (MgSO₄) and concentrated to an oil (0.92 g).

The oil was dissolved in anhydrous toluene (40 mL). Meptazinol free base (1.05 g, 4.5 mmol) was then added, and the resulting solution was heated at reflux for 4 hours. The solvent was partially evaporated and the residue was purified by medium-pressure chromatography on silica. The resulting product was eluted with ethyl acetate containing 0.1% triethylamine, to afford 1.50 g of a viscous, colorless oil, R_{f} 0.35 (ethyl acetate—triethylamine, 99.9:0.1).

The purified material (0.75 g, 1.74 mmol) was dissolved in trifluoroacetic acid (7 mL) and the resulting solution was stirred at room temperature for 2 hours, and then evaporated to dryness. Residual trifluoroacetic acid was removed by addition of chloroform to the residue and evaporation (repeated five times). The residue was dried under high vacuum at 60° C. for 3 hours to afford meptazinol-(S)-valine carbamate trifluoroacetate, as a gum.

¹H NMR (DMSO-d₆, 300 MHz): δ 8.02 (m, 1H, NH), 7.43-7.36 (m, 1H, ArH), 7.27-7.15 (m, 2H, 2×ArH), 7.06-7.00 (m, 1H, ArH), 4.01-3.89 (m, 2H, CH₂N), 3.47-3.40 (m, 1H, α-CH), 3.24-3.01 (m, 2H, CH₂N), 2.91+2.85 (m, 3H, CH₃N), 2.13 (m, 1H, β-CH), 1.95-1.40 (m, 8H, 4×CH₂), 0.96 (m, 6H, 2× isopropyl CH₃), 0.54 (m, 3H, CH₃).

LC-MS: m/z=377. Consistent for the protonated parent molecule (MH⁺).

### Example 3

### Synthesis of Meptazinol-(S)-Valine Carbamate Hydrochloride

Meptazinol-(S)-valine carbamate hydrochloride was synthesized as shown in Scheme 4, below.

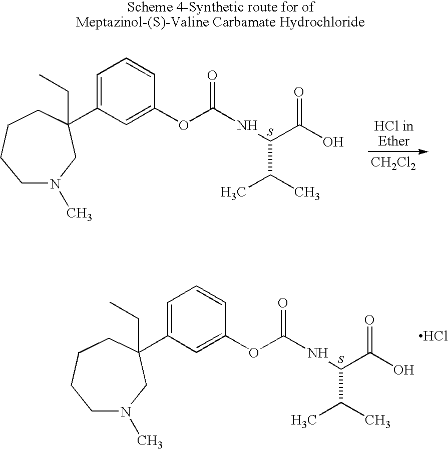

Initially meptazinol-(S)-valine carbamate zwitterion was dissolved in dichloromethane and a solution of hydrogen chloride in diethyl ether was added. The corresponding hydrochloride salt was obtained as a glassy solid following removal of the solvents.

### Example 4

### Synthesis of Meptazinol-(S)-Valine-(S)-Valine Carbamate

The synthetic route for meptazinol-(S)-valine-(S)-valine carbamate is shown in scheme 5, below.

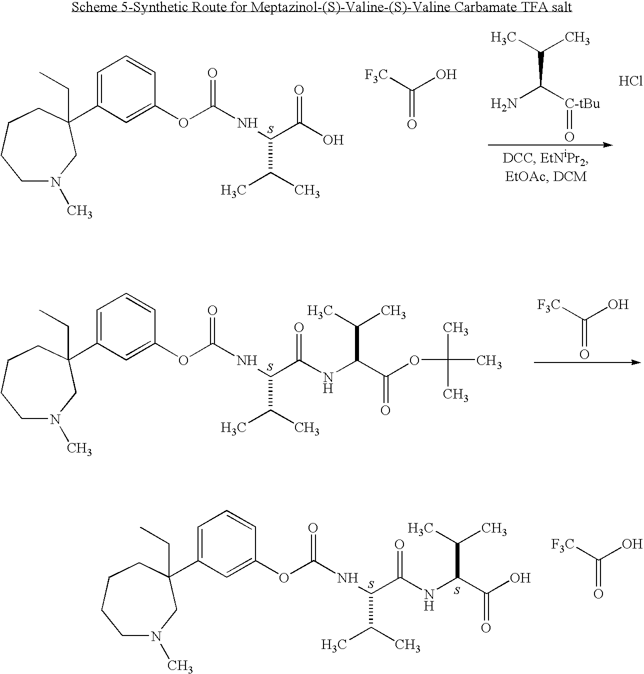

To meptazinol (S)-valine carbamate trifluoroacetate (1.30 g, 2.65 mmol), (S)-valine tert-butyl ester hydrochloride (0.67 g, 3.21 mmol) and ethyl di-isopropylamine (0.56 mL, 3.21 mmol, 0.41 g) in a stirred mixture of anhydrous dichloromethane (6 mL) and ethyl acetate (6 mL) cooled in an ice-bath under nitrogen was added dicyclohexylcarbodi-imide (0.66 g, 3.21 mmol) portionwise. The reaction mixture was allowed to warm to room temperature and stirred overnight. Ethyl acetate was added and the mixture was filtered through Celite. The solvent was evaporated and the residue was purified by medium-pressure chromatography on silica, eluting with 94.9% ethyl acetate-5% methanol-0.1% triethylamine to afford 0.75 g of a viscous, colorless oil, R_{f} 0.19 (ethyl acetate-methanol, 9:1 plus trace Et₃N).

The purified material (0.75 g, 1.41 mmol) was dissolved in trifluoroacetic acid (10 mL) and the resulting solution was stirred at room temperature for 2 hours, after which the trifluoroacetic acid was evaporated. Residual trifluoroacetic acid was removed by addition of chloroform to the residue and evaporation (repeated five times). The residue was dried under high vacuum at 60° C. for 3 hours to afford meptazinol-(S)-valine-(S)-valine carbamate trifluoroacetate (0.83 g, 100%), as a viscous oil.

¹H NMR (DMSO-d₆, 300 MHz): δ 8.50 (broad s, 1H, NH), 8.00 (d, J=8.7 Hz, 1H, NH), 7.81 (d, J=8.7 Hz, 1H, ArH), 7.42-7.38 (m, 1H, ArH), 7.27-7.14 (m, 1H, ArH), 7.05-7.01 (m, 1H, ArH), 4.19-3.98 (m, 2H, CH₂N), 3.48-3.41 (m, 2H, 2× valine α-CH), ca. 3.2-3.0 (m, 2H, CH₂N), 2.90+2.84 (m, 3H, CH₃N), 2.05-1.46 (m, 10H, 4×CH₂+2× valine P—CH), 0.91 (m, 12H, 4× isopropyl CH₃), 0.52 (m, 3H, CH₃).

LC-MS: m/z=476.69. Consistent for protonated parent ion (MH⁺).

### Example 5

### Synthesis of Oxymorphone (S)-Valine Carbamate Hydrochloride

The synthetic route for oxymorphone-(S)-valine carbamate hydrochloride is shown in scheme 6, below.

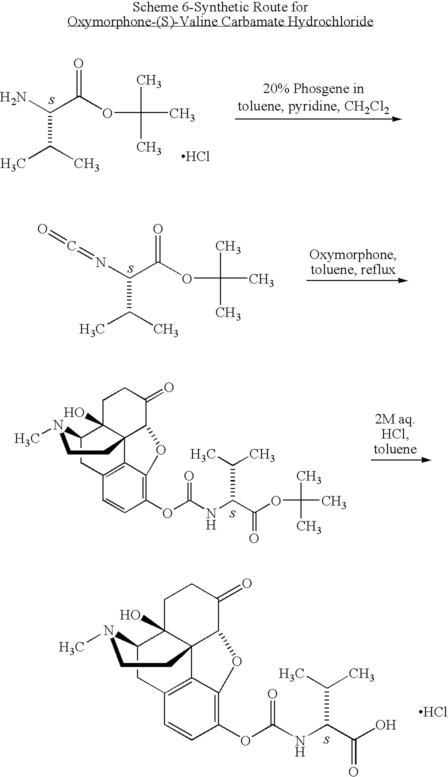

A suspension of (S)-valine tert-butyl ester hydrochloride (2.20 g, 10.5 mmol) and pyridine (3.37 mL, 42.0 mmol, 3.30 g) in anhydrous dichloromethane (60 mL) was cooled in an ice-bath under nitrogen. Next, 20% phosgene in toluene (7.35 mL, 14.0 mmol, 6.90 g) was added dropwise to the stirred mixture. Stirring was continued for a further 2 hours while the reaction was allowed to warm to room temperature. The resulting mixture was diluted with more dichloromethane, and washed with ice-cold 1M hydrochloric acid, followed by brine and was then dried (MgSO₄) and concentrated to give an oil (2.0 g).

The oil was dissolved in anhydrous toluene (50 mL) and oxymorphone free base (1.97 g, 6.56 mmol) was added and the solution. The solution was then heated at reflux for 4 hours (the oxymorphone was not initially soluble in toluene but dissolved slowly as the reaction proceeded). The solvent was then evaporated and the residue purified by medium-pressure chromatography on silica, eluting with a solvent gradient of 4→20% methanol in dichloromethane containing 0.1% triethylamine, to afford oxymorphone-(S)-valine carbamate tert-butyl ester (2.45 g, 75%), as a glassy solid.

R_{f} 0.51 (4:1 dichloromethane-methanol plus trace of triethylamine).

A portion of this material (1.87 mg, 3.74 mmol) was dissolved in toluene (30 mL) and 2M hydrochloric acid (30 mL) was added, resulting in a two-phase mixture. The mixture was vigorously stirred and heated at 45° C. (sealed flask) overnight, and then allowed to cool. The mixture was diluted with additional toluene and water and the layers separated. The aqueous layer was washed once with diethyl ether and then freeze-dried to afford oxymorphone-(S)-valine carbamate hydrochloride (1.61 g, 90%), as an off-white powder.

¹H NMR (DMSO-d₆, 300 MHz): δ 9.40 (broad s, 1H, oxymorphone OH), 8.17 (d, J=8.4 Hz, 1H, carbamate NH), 6.95 (d, J=8.1 Hz, 1H, ArH), 6.81 (d, J=8.1 Hz, 1H, ArH), 5.14 (s, 1H, CH—O—Ar), 3.87 (dd, J=8.1, 6.0 Hz, 1H, valine α-CH), 3.76 (m, 1H, CHN), 3.45 (d, J=20.1 Hz, 1H, ½×CH₂N), 3.16-2.90 (m, 4H, benzylic CH₂+½×CH₂N+½×CH₂), 2.85 (s, 3H, CH₃N), 2.67 (m, 1H, ½×CH₂), 2.11 (m, 2H, valine β CH+½×CH₂), 1.97 (d, J=13.8 Hz, 1H, ½×CH₂), 1.48 (t, J=13.8 Hz, 2H, CH₂), 0.93 (d, J=6.9 Hz, 6H, 2× valine CH₃).

LC-MS: Single peak m/z=445.10; consistent for protonated parent ion.

### Example 6

### Synthesis of Buprenorphine-(S)-Valine Carbamate

The synthetic route for buprenorphine-(S)-valine carbamate is given in Scheme 7, below.

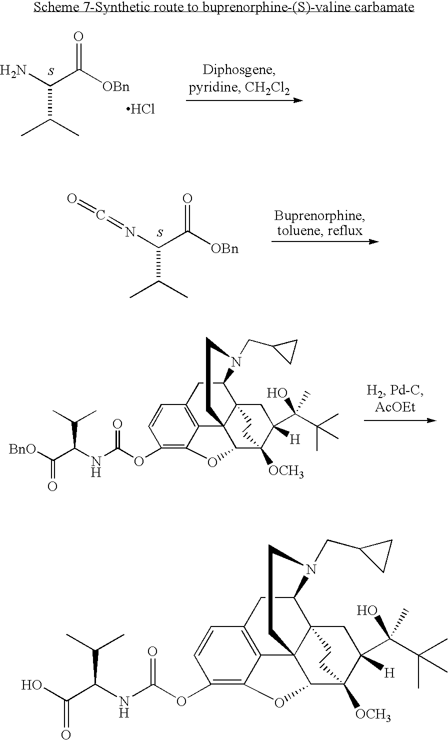

A suspension of (S)-valine benzyl ester hydrochloride (1.00 g, 2.21 mmol) and pyridine (1.34 mL, 1.29 g, 16.41 mmol) in anhydrous dichloromethane (40 mL) was cooled in an ice-bath under nitrogen. Next, diphosgene (0.32 mL, 528 mg, 2.67 mmol) was added dropwise to the stirred mixture. Stirring was continued for a further 2 hours while the reaction was allowed to warm to room temperature. The resulting mixture was diluted with more dichloromethane and then washed with ice-cold 1M hydrochloric acid, followed by brine. The mixture was dried (MgSO₄) and concentrated to give a yellow oil (0.90 g).

Buprenorphine (500 mg, 1.07 mmol) was suspended in anhydrous toluene (15 mL). A solution of (S)-valine benzyl ester isocyanate (0.75 g, 3.21 mmol) in toluene (10 mL) was added and the solution was heated at reflux overnight (the buprenorphine was not initially soluble in toluene but dissolved once the reflux temperature was achieved). The solvent was evaporated and the residue purified by medium-pressure chromatography on silica (petrol-ethyl acetate 9:1) to afford buprenorphine valine carbamate benzyl ester as a glassy solid (398 mg, 53%).

A solution of this material (398 mg, 0.64 mmol) in ethyl acetate (5 mL) was added to a suspension of 10% palladium-carbon (99 mg) in ethyl acetate (5 mL). The mixture was stirred under a hydrogen atmosphere for 5 hours and was then filtered through celite. The solvent was evaporated and the residue purified by medium-pressure chromatography on silica, eluting with a gradient of 3→10% methanol in dichloromethane, to afford buprenorphine valine carbamate (92 mg, 26%), as a white solid.

¹H NMR (DMSO-d₆, 300 MHz): δ 12.46 (br s, 1H, OH), 7.88 (d, J=8.4 Hz, 1H, NH), 6.71 (d, J=8.1 Hz, 1H, ArH), 6.50 (d, J=8.1 Hz, 1H, ArH), 6.19 (d, J=8.7 Hz, 1H, CH), 5.37 (s, 1H, CH), 4.34 (s, 1H, CH), 3.90 (m, 1H, α-H), 3.70 (m, 2H, CH), 3.27 (s, 3H, OMe), 2.86 (br d, J=18.6 Hz, 1H, CH), 2.66 (m, 1H, CH), 2.50 (br s, 1H, CH), 2.16 (m, 3H, CH), 2.08-1.82 (m, 4H, CH), 1.62-1.48 (m, 4H, CH), 1.15 (s, 3H, Methyl), 0.99 (m, 1H, CH), 0.85 (s, 9H, tert-butyl), 0.75 (m, 12H, isopropyl CH₃ and CH), 0.34 (m, 3H, 3× cyclopropyl CH), 0.00 (br s, 2H, 2× cyclopropyl CH).

LC-MS: Single peak m/z=611.20; Consistent for protonated ion.

### Example 7

### Synthesis of Nalbuphine-(S)-Valine Carbamate

Nalbuphine Carbamate Prodrug Synthesis, in General

The synthesis of nalbuphine amino-acid carbamate can be achieved in two distinct steps. Essentially, an (S)-amino acid tert-butyl ester hydrochloride can be treated with diphosgene in the presence of pyridine, and the resulting isocyanate can be used immediately in the next reaction step. Reaction with nalbuphine free-base in refluxing toluene for four hours affords, after purification by column chromatography (to remove the minor 6-O-regioisomer), nalbuphine-(S)-amino acid carbamate tert-butyl ester. Subsequent deprotection can be achieved using a suitable acid such as trifluoroacetic acid or hydrochloric acid, to give the desired nalbuphine amino-acid carbamate as the corresponding acid salt.

Nalbuphine-(S)-Valine Carbamate

A suspension of an (S)-valine tert-butyl ester hydrochloride (1 mol equiv.) and pyridine (4 mol equiv.) in anhydrous dichloromethane is cooled in an ice-bath under nitrogen. A 20% solution of phosgene in toluene (0.6 mol equiv.) is then added dropwise to the stirred mixture. Stirring is continued for a further period of 2 hours while the reaction is allowed to warm to room temperature. The resulting mixture is diluted with more dichloromethane and washed with an ice-cold solution of 1M hydrochloric acid, followed by brine. The product is then dried (MgSO₄) and concentrated.

The residue is dissolved in anhydrous toluene. Nalbuphine free base (1 mol equiv.) is then added, and the solution is heated at reflux for 4 hours. The solvent is partially evaporated and the residue purified by medium-pressure chromatography on silica to afford the 3-O-substituted nalbuphine-(S)-valine carbamate tert-butyl ester.

The nalbuphine-(S)-valine carbamate tert-butyl ester is dissolved in an excess of trifluoroacetic acid and stirred at room temperature for 30 minutes. After this time, the solution is evaporated to dryness to afford the required 3-O-substitued nalbuphine-(S)-amino acid carbamate trifluoroacetate. These process steps are shown in Scheme 8.

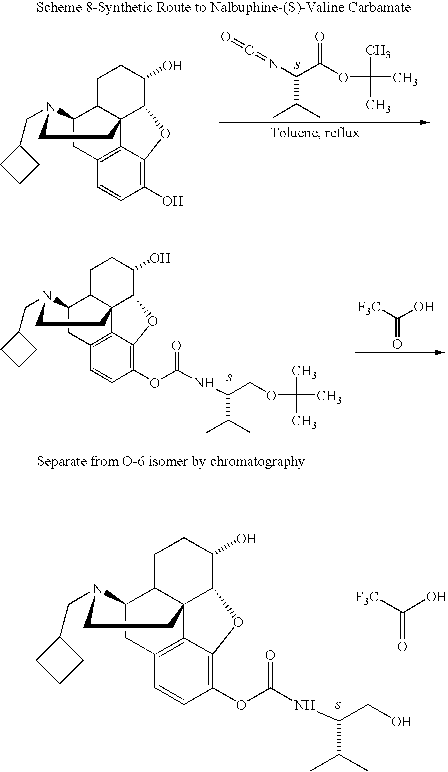

### Example 8

### In Vitro Assessment of Stability of Various Opioid Amino Acid Carbamates Under Conditions Prevailing in the Gut

Methodology

Inherent chemical and biological stability of the prodrug under the conditions prevailing in the GI tract is a mandatory requirement. If the prodrug should be prematurely hydrolyzed it would negate the opportunity to deliver, systemically, the intact prodrug from which the active drug may be continuously generated. To investigate this various opioid amino acid valine carbamate were incubated at 37° C. in simulated gastric and simulated intestinal juice (USP defined composition) for 2 hours. The remaining concentration of the prodrug was assayed by HPLC. For comparative purposes stabilities in three other standard media were also determined.

Results

TABLE 2
Prodrug Stability in Various Media

| | Simulated | Simulated | | |
|---|---|---|---|---|
| | gastric fluid | intestinal fluid | Distilled water | |
| | (pH 1.1): | (pH 6.8): | (pH 5.9): | pH 10.0 buffer: |
| | % remaining | % remaining | % remaining | % remaining |
| Compound | after 2 h/37° C. | after 2 h/37° C. | after 2h/20° C. | after 2 h/20° C. |
| Meptazinol-Val- | 100 | 99 | 100 | 66 |
| Carbamate | | | | |
| Oxymorphone-Val- | 100 | 97 | 100 | 32 |
| Carbamate | | | | |
| Oxymorphone-Phe- | 100 | 82 | 100 | 17 |
| Carbamate | | | | |
| Oxymorphone-Isoleu- | 100 | 97 | 100 | 36 |
| Carbamate | | | | |
| Oxymorphone-Methio- | 100 | 96 | 100 | 8 |
| Carbamate | | | | |
| Buprenorphine-Val- | 100 | 98 | 100 | 74 |
| Carbamate | | | | |
| Buprenorphine-Phe- | 100 | 45 | 99 | 74 |
| Carbamate | | | | |
| Buprenorphine-Asp- | 100 | 99 | 100 | 85 |
| Carbamate | | | | |

As can be seen in Table 2, these opioid amino acid carbamate prodrugs are generally inherently stable under the conditions existing in the GI tract. One apparent exception is the phenylalanine carbamate of buprenorphine. However, overall, these compounds appear to be stable, and would be expected to be absorbed intact.

### Example 9

### Bioavailability of Meptazinol from Various Meptazinol Amino Acid Conjugates in the Dog

Test substances (i.e., meptazinol and various meptazinol amino acid carbamates) were administered by oral gavage to one of two groups of five dogs in a multiway crossover design. The characteristics of the test animals are set out in Table 3.

TABLE 3
Characteristics of experimental dogs used in study

| | |
|---|---|
| Species | Dog |
| Type | Beagle |
| Number and sex | 5 males |
| Approximate age | 3-4 months at the start of treatment |
| Approx. bodyweight | 7-9 kg at the start of treatment |
| Source | Huntingdon Life Sciences stock |

Blood samples were taken at various times after administration and submitted to analysis for the parent drug and pro-drug using a validated LC-MS-MS assay. Pharmacokinetic parameters derived from the plasma analytical data were determined using Win Nonlin. The results are given in Table 4.

TABLE 4
Comparative pharmacokinetics of meptazinol from various
meptazinol prodrugs following their oral administration
at 1 mg meptazinol free base/kg to groups of five dogs

| | Cmax | AUC | |
|---|---|---|---|
| | (mean ng/ | (mean ngh/ | Mean relative |
| Compound name | mL ± SD) | mL ± SD) | bioavailability % |
| Meptazinol HCl (oral) - | 5.28 ± 1.5 | 15.6 ± 7.4 | — |
| Group 1 (G1) | | | |
| Meptazinol HCl (oral) - | 2.57 ± 0.63 | 12.8 ± 3.4 | — |
| Group 2 (G2) | | | |
| Meptazinol tyrosine | 2.23 ± 0.16 | 6.61* ± 0.70 | 52 |
| carbamate TFA | | | |
| salt (G2) | | | |
| Meptazinol leucine | 3.32 ± 1.4 | 9.27 ± 1.6 | 70 |
| carbamate TFA salt | | | |
| (G1) | | | |
| Meptazinol | 3.5 ± 0.4 | 14.2 ± 5.5 | 75 |
| phenylalanine methyl | | | |
| ester HCl (G1) | | | |
| Meptazinol glycine | 2.51 ± 0.90 | 10.2* ± 3.4 | 79 |
| carbamate TFA salt (G2) | | | |
| Meptazinol ala-ala | 2.73 ± 1.28 | 11.4 ± 2.9 | 79 |
| dipeptide TFA salt (G1) | | | |
| Meptazinol alanine | 5.2 ± 1.59 | 16.3 ± 3.6 | 116 |
| carbamate HCl salt (G2) | | | |
| Meptazinol tryptophan | 8.21 ± 2.56 | 19.1* ± 8.9 | 149 |
| carbamate TFA salt (G2) | | | |
| Meptazinol isoleucine | 19.6 ± 8.6 | 34.9 ± 11 | 251 |
| carbamate TFA salt (G1) | | | |
| Meptazinol val-ala | 16.4 ± 7.5 | 36.5 ± 13 | 260 |
| dipeptide TFA salt (G1) | | | |
| Meptazinol apartic | 2.65 ± 0.34 | 34.8* ± 1.0 | 273 |
| acid carbamate TFA | | | |
| salt (G2) | | | |
| Meptazinol val-gly | 18.1 ± 9.4 | 39.9 ± 17 | 294 |
| dipeptide TFA salt (G1) | | | |
| Meptazinol val-val | 18.7 ± 8.1 | 45.0 ± 12 | 330 |
| dipeptide TFA salt (G1) | | | |
| Meptazinol methionine | 7.22 ± 1.68 | 55.4* ± 4.40 | 433 |
| carbamate TFA salt (G2) | | | |
| Meptazinol valine | 31.1 ± 14.7 | 62.4 ± 20.4 | 442 |
| carbamate camsylate | | | |
| (G1) | | | |
| Meptazinol valine | 37.3 ± 13.6 | 67.7 ± 25 | 483 |
| carbamate TFA salt (G1) | | | |
| Meptazinol valine | 24.8 ± 1.4 | 67.6 ± 9.2 | 520 |
| carbamate HCl (G2) | | | |
| Meptazinol valine | 44.8 ± 8.2 | 78.1 ± 17.1 | 606 |
| carbamate methyl | | | |
| ester HCl (G2) | | | |
| Meptazinol valine | 28.4 ± 14.1 | 84.9 ± 40.6 | 669 |
| carbamate zwitterion | | | |
| (G2) | | | |

G1 = group 1 dogs, G2 = group 2 dogs, *AUC0-t

Table 4 shows the mean meptazinol Cₘₐₓ, AUC and relative bioavailability of meptazinol after administration of various carbamate prodrugs. It is evident that those containing valine demonstrated the most significant improvements in oral bioavailabilities compared to meptazinol itself. Interestingly the other carbamate conjugate which performed well was that with methionine.

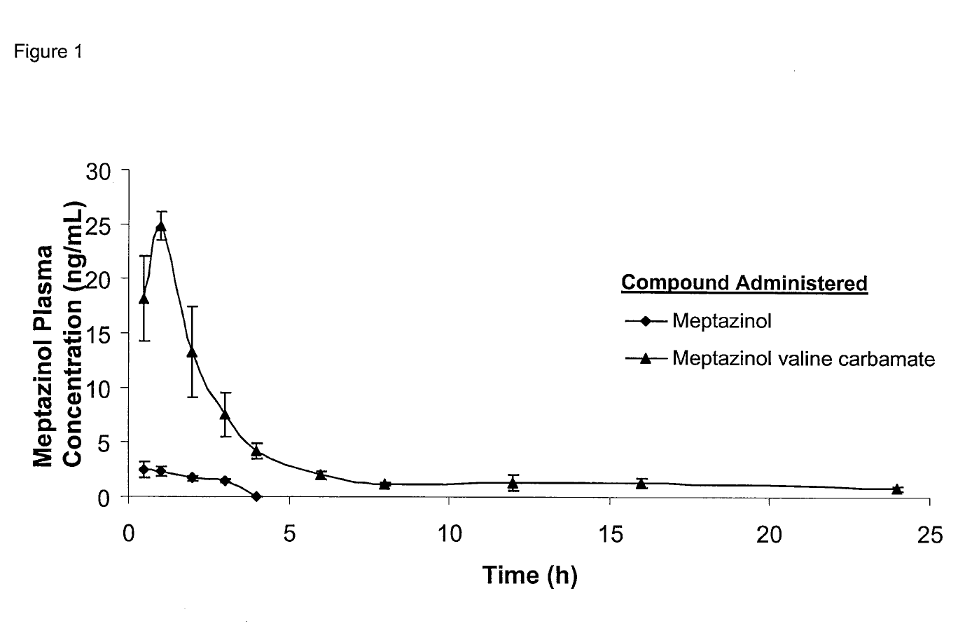

Tables 5 and 6 and FIG. 1 present more PK data after oral administration of either meptazinol itself or its valine carbamate conjugate to dogs. From these data, it can be seen that there was a very substantial increase in bioavailability with the systemic exposure to the drug (expressed by the mean AUC), increasing from 12.8±3.4 ng.h/mL to 67.6±9.2. Not only does this represent >5-fold increase in oral bioavailability for meptazinol when administered as a prodrug, the prodrug also shows less variability in meptazinol serum levels, as compared to administration of meptazinol itself. The relative standard deviations for meptazinol and prodrug were 26% and 14%, respectively.

TABLE 5
Pharmacokinetics of meptazinol and meptazinol glucuronide after oral administration
of meptazinol HCl at 1 mg meptazinol free base equivalents/kg

| Pharmacokinetic | Dog No. | | | | | | |
|---|---|---|---|---|---|---|---|
| parameter | 1 | 2 | 3 | 4 | 5 | Mean | sd |
| Meptazinol | | | | | | | |
| Cₘₐₓ (ng/mL) | 2.53 | 1.86 | 2.28 | 3.57 | 2.59 | 2.57 | 0.63 |
| Tₘₐₓ (h) | 1 | 1 | 0.5 | 0.5 | 0.5 | 0.5^{a} | |
| AUCₜ (ng · h/mL) | 5.87 | 6.25 | 4.98 | 7.88 | 6.35 | 6.27 | 1.05 |
| AUC (ng · h/mL) | 11.4 | 18.8 | 11.6 | 11.3 | 10.7 | 12.8 | 3.4 |
| t½ (h) | 2.6 | 7.8 | 3.4 | 2.2 | 2.9 | 3.1^{b} | |
| F^{c} (%) | 4.2 | 7.0 | 4.3 | 4.2 | 4.0 | 4.7 | 1.3 |
| Meptazinol | | | | | | | |
| glucuronide | | | | | | | |
| Cₘₐₓ (ng/mL) | 1700 | 1820 | 1790 | 3300 | 2530 | 2230 | 690 |
| Tₘₐₓ (h) | 2 | 1 | 1 | 0.5 | 0.5 | 0.75^{a} | |
| AUCₜ (ng · h/mL) | 6410 | 6970 | 5800 | 9470 | 7290 | 7190 | 1390 |
| AUC (ng · h/mL) | 6430 | 6990 | 5850 | 9490 | 7300 | 7220 | 1390 |
| t½ (h) | 3.2 | 3.0 | 4.0 | 3.2 | 2.7 | 3.2^{b} | |

TABLE 6
Pharmacokinetics of meptazinol and meptazinol glucuronide
in dogs after oral administration of meptazinol valine carbamate
HCl at 1 mg meptazinol free base equivalents/kg

| Pharmacokinetic | Dog No. | | | | | | |
|---|---|---|---|---|---|---|---|
| parameter | 1 | 2 | 3 | 4 | 5 | Mean | sd |
| Meptazinol | | | | | | | |
| Cₘₐₓ (ng/mL) | 23.8 | 23.0 | 25.9 | 25.6 | 25.9 | 24.8 | 1.4 |
| Tₘₐₓ (h) | 1 | 1 | 1 | 1 | 1 | 1^{a} | |
| AUC (ng · h/mL) | 53.3 | 73.9 | 56.3 | 75.2 | 71.2 | 66.0 | 10.4 |
| AUC (ng · h/mL) | 57.8 | 77.0^{c} | 58.0^{c} | 70.2^{c} | 75.1 | 67.6 | 9.2 |
| t½ (h) | 1.7 | 3.6^{c} | 5.2^{c} | 4.7^{c} | 2.1 | 1.8^{b} | |
| T_{>50% Cmax} (h) | 0.5 | 1.5 | 0.5 | 0.5 | 1.5 | 0.5^{a} | |
| F^{d} (%) | 21.5 | 28.6 | 21.6 | 26.1 | 27.0 | 25.0 | 3.2 |
| Meptazinol | | | | | | | |
| glucoronide | | | | | | | |
| Cₘₐₓ (ng/mL) | 135 | 132 | 141 | 205 | 181 | 159 | 33 |
| Tₘₐₓ (h) | 3 | 3 | 3 | 3 | 3 | 3^{a} | |
| AUCₜ (ng · h/mL) | 1180 | 1340 | 1640 | 2100 | 1300 | 1510 | 370 |
| AUC (ng · h/mL) | 1230 | 1390 | 1910^{c} | 2440 | 1320 | 1600 | 570 |
| t½ (h) | 5.0 | 4.2 | 8.6^{c} | 8.2 | 3.6 | 4.8^{b} | |

^{a}Median value
^{b}Calculated as ln2/mean k
^{c}The correlation coefficient was ≦0.75,
^{d}Calculated using mean AUC value following iv administration to Group 1 dogs

### Example 10

### Bioavailability of Oxymorphone from Various Oxymorphone Amino Acid Conjugates in the Dog

Test substances (i.e., oxymorphone and various oxymorphone amino acid carbamates) were administered by oral gavage to a group of five dogs in a multiway-way crossover design. The characteristics of the test animals are set out in Table 7.

TABLE 7
Characteristics of experimental dogs used in study

| | |
|---|---|
| Species | Dog |
| Type | Beagle |
| Number and sex | 5 males |
| Approximate age | 3-4 months at the start of treatment |
| Approx. bodyweight | 7-9 kg at the start of treatment |
| Source | Huntingdon Life Sciences stock |

Blood samples were taken at various times after administration and submitted to analysis for the parent drug and pro-drug using a validated LC-MS-MS assay. Pharmacokinetic parameters derived from the plasma analytical data were determined using Win Nonlin. The results are given in Table 8.

TABLE 8
Comparative pharmacokinetics of oxymorphone following oral
administration of either the drug itself or various prodrugs
at 0.5 mg oxymorphone free base/kg to groups of five dogs

| | Cmax | AUC | |
|---|---|---|---|
| | (mean ng/ | (mean ngh/ | Mean relative |
| Compound name | mL ± SD) | mL ± SD) | bioavailability % |
| Oxymorphone HCl | 3.8 ± 0.77 | 12.2 ± 1.6 | — |
| Oxymorphone tyrosine | 4.62 ± 1.12 | 17.3 ± 3.0 | 157 |
| carbamate TFA | | | |
| Oxymorphone | 5.45 ± 1.32 | 29.5 ± 2.2 | 244 |
| phenylalanine carbamate | | | |
| HCl | | | |
| Oxymorphone | 13.8 ± 4.6 | 55.4 ± 15.2 | 436 |
| methionine carbamate | | | |
| TFA | | | |
| Oxymorphone glycine | 21 ± 10.9 | 67.9 ± 34.1 | 554 |
| carbamate TFA | | | |
| Oxymorphone isoleucine | 17.2 ± 9.9 | 65.8 ± 32 | 570 |
| carbamate TFA | | | |
| Oxymorphone valine | 14.6 ± 8.69 | 51.2 ± 35 | 816 |
| carbamate TFA* | | | |
| Oxymorphone valine | 21.6 ± 7.2 | 94.4 ± 33.3 | 816 |
| carbamate HCl** | | | |

*Different group of dogs
**N = 4 dogs

Table 8 shows that mean oxymorphone Cₘₐₓ, AUC and relative bioavailability of oxymorphone after administration of various carbamate prodrugs. It is evident that the valine carbamate demonstrated the most significant improvement in oral bioavailability—some 8-fold greater—compared to oxymorphone itself. Another carbamate conjugate which performed well was the structurally related amino acid isoleucine. The isoleucine prodrug showed a 6.5 fold improvement in oral bioavailability. The glycine carbamate also showed a significant improvement in bioavailability.

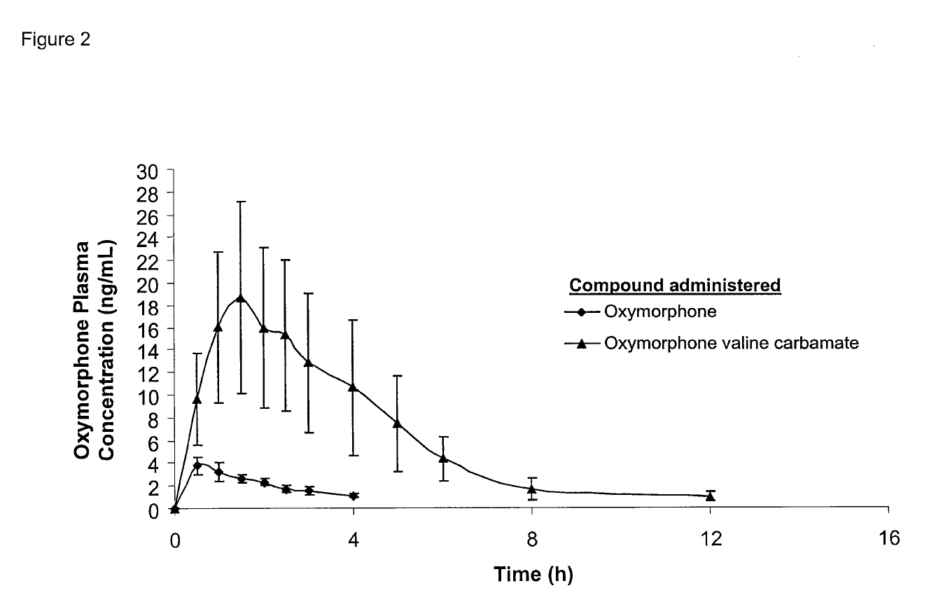

Tables 9 and 10 and FIG. 2 present more detailed PK data of oxymorphone after oral administration to dogs of either oxymorphone itself or its valine carbamate conjugate. Peak plasma levels of oxymorphone were clearly very much higher after giving an equimolar dose of the oxymorphone prodrug compared to oxymorphone itself. Indeed, Cₘₐₓ values were some 6-fold higher for the prodrug while overall exposure, as reflected in the AUC, was approximately 8-fold greater.

Furthermore, while peak oxymorphone plasma levels after giving the prodrug were still reached relatively quickly, (within 2 hr.) ensuring a rapid onset of action, plasma oxymorphone concentrating were subsequently maintained over an extended period, compared to drug levels after giving oxymorphone itself. This was reflected in the time for which plasma drug levels were maintained above 50% of the Cₘₐₓ values which was almost twice as long after the prodrug than after giving the drug itself. This maintenance of plasma concentrations after giving the prodrug, enabling less frequent dosing while still sustaining of analgesia, may be the result of continuing generation of the drug from a plasma reservoir of the prodrug of which there is substantial presence. Plasma levels were approx. 4-fold greater than the drug itself which may provide a source for continued production of the pharmacologically active species.

TABLE 9
Pharmacokinetic of oxymorphone in dogs orally administered
oxymorphone HCl at 0.5 mg free base equivalents/kg

| Pharmacokinetic | Dog No. | | | | | | |
|---|---|---|---|---|---|---|---|
| parameter | 1 | 2 | 3 | 4 | 5 | Mean | sd |
| Cₘₐₓ (ng/mL) | 3.96 | 3.13 | 5.03 | 3.20 | 3.68 | 3.80 | 0.77 |
| Tₘₐₓ (h) | 0.5 | 1 | 0.5 | 0.5 | 0.5 | 0.5^{a} | |
| AUCₜ (ng · h/mL) | 8.76 | 6.78 | 9.91 | 6.96 | 9.66 | 8.41 | 1.47 |
| AUC (ng · h/mL) | 11.1 | 10.6 | 14.3 | 11.8 | 13.4 | 12.2 | 1.6 |
| t½ (h) | 1.7 | 2.3 | 2.8 | 3.1 | 2.0 | 2.2^{b} | |
| T_{>50% Cmax} (h) | 1.5 | 2.0 | 1.5 | 1.5 | 2.5 | 1.5^{a} | |
| F_{absolute} (%)^{c} | 3.8 | 3.6 | 4.9 | 4.0 | 4.6 | 4.2 | 0.5 |

^{a}Median value
^{b}Calculated as ln2/mean k
^{c}Calculated using intravenous AUC value of 146 ng · h/mL

TABLE 10
Pharmacokinetic of oxymorphone and its valine carbamate after oral administration
of the prodrug HCl at 0.5 mg oxymorphone free base equivalents/kg

| Pharmacokinetic | Dog No. | | | | | | | |
|---|---|---|---|---|---|---|---|---|
| parameter | 1 | 2 | 3 | 4 | 5 | Mean | | sd |
| Oxymorphone | | | | | | | | |
| Cₘₐₓ (ng/mL) | 16.9 | 31.0 | 15.1 | 23.3 | 8.18 | 18.9 | (21.6) | 8.6 (7.2) |
| Tₘₐₓ (h) | 1.5 | 1.5 | 1.5 | 2.5 | 1.5 | 1.5^{a} | (1.5) | |
| AUCₜ (ng · h/mL) | 61.7 | 125 | 61.2 | 115 | 33.2 | 79.2 | (90.7) | 39.1 (34.1) |
| AUC (ng · h/mL) | 66.9 | 128 | 64.5 | 118 | 36.7 | 82.8 | (94.4) | 38.7 (33.3) |
| t½ (h) | 1.1 | 3.0 | 2.7 | 2.7 | 2.2 | 2.1^{b} | (2.1) | |
| T_{>50% Cmax} (h) | 3 | 2.5 | 2.5 | 3 | 2.5 | 2.5^{a} | (2.75) | |
| F_{absolute} (%)^{c} | 22.9 | 43.8 | 22.1 | 40.4 | 12.6 | 28.4 | (32.3) | 13.3 (11.4) |
| Fᵣₑₗₐₜᵢᵥₑ (%) | 603 | 1208 | 451 | 1000 | 274 | 707 | (816) | 387 (349) |

| | | | | | | | |
|---|---|---|---|---|---|---|---|
| Oxymorphone valine | | | | | | | |
| carbamate | | | | | | | |
| Cₘₐₓ (ng/mL) | 75.0 | 96.5 | 96.0 | 84.5 | 88.5 | 88.1 | 8.9 |
| Tₘₐₓ (h) | 1 | 1 | 0.5 | 1 | 1.5 | 1^{a} | |
| AUCₜ (ng · h/mL) | 232 | 330 | 300 | 314 | 316 | 298 | 39 |
| AUC (ng · h/mL) | 234 | 336 | 317 | 336 | 331 | 311 | 44 |
| t½ (h) | 2.4 | 2.8 | 4.5 | 6.8 | 1.7 | 2.9^{b} | |
| T_{>50% Cmax} (h) | 2 | 1.5 | 1.5 | 2.5 | 2.5 | 2^{a} | |

^{a}Median value
^{b}Calculated as ln2/mean k
^{c}Calculated using intravenous AUC value of 146 ng · h/mL
Mean, median and sd values in parentheses calculated excluding Dog 5

### Example 11

### Bioavailability of Buprenorphine from Various Buprenorphine Amino Acid Conjugates in the Dog

Test substances (i.e., buprenorphine and various buprenorphine amino acid carbamates) were administered by oral gavage to two groups of five dogs in a multiway-way crossover design. The characteristics of the test animals are set out in Table 11.

TABLE 11
Characteristics of experimental dogs used in study

| | |
|---|---|
| Species | Dog |
| Type | Beagle |
| Number and sex | 5 males |
| Approximate age | 3-4 months at the start of treatment |
| Approx. bodyweight | 7-9 kg at the start of treatment |
| Source | Huntingdon Life Sciences stock |

Blood samples were taken at various times after administration and submitted to analysis for the parent drug and pro-drug using a validated LC-MS-MS assay. Pharmacokinetic parameters derived from the plasma analytical data were determined using Win Nonlin. The results are given in Table 12.

TABLE 12
Comparative pharmacokinetics of buprenorphine following administration
of either the drug itself or various prodrugs at 0.5 mg buprenorphine
free base/kg to groups of five dogs

| | Cmax | AUC | |
|---|---|---|---|
| | (mean ng/ | (mean ngh/ | Mean relative |
| Compound name | mL ± SD) | mL ± SD) | bioavailability % |
| Buprenorphine HCl | 2.88 ± 1.58 | 3.19 ± 1.63 | — |
| (group1) | | | |
| Buprenorphine HCl | 2.65 ± 0.80 | 12.3 ± 5.8 | — |
| (group2) | | | |
| Buprenorphine glycine | 0.35 ± 0.20 | 1.20 ± 1.1 | 35.4 |
| carbamate (group1) | | | |
| Buprenorphine lysine | 0.25 ± 0.19 | 4.70 | 38 |
| carbamate (group2) | | | |
| Buprenorphine | 0.86 ± 0.32 | 9.66 ± 1.92 | 84.5 |
| phenylalanine | | | |
| carbamate (group2) | | | |
| Buprenorphine leucine | 1.68 ± 0.56 | 13.1 ± 3.5 | 106 |
| carbamate (group2) | | | |
| Buprenorphine | 1.25 ± 0.51 | 13.6 ± 3.9 | 110 |
| isoleucine carbamate | | | |
| (group2) | | | |
| Buprenorphine valine | 3.08 ± 0.67 | 23.2 ± 11.3 | 883 |
| carbamate (group1) | | | |

Table 12 shows that mean buprenorphine Cₘₐₓ, AUC and relative bioavailability of buprenorphine after administration of various carbamate prodrugs. It is evident that the valine carbamate demonstrated the most significant improvement in oral bioavailabilities—some 9-fold greater—compared to buprenorphine itself.

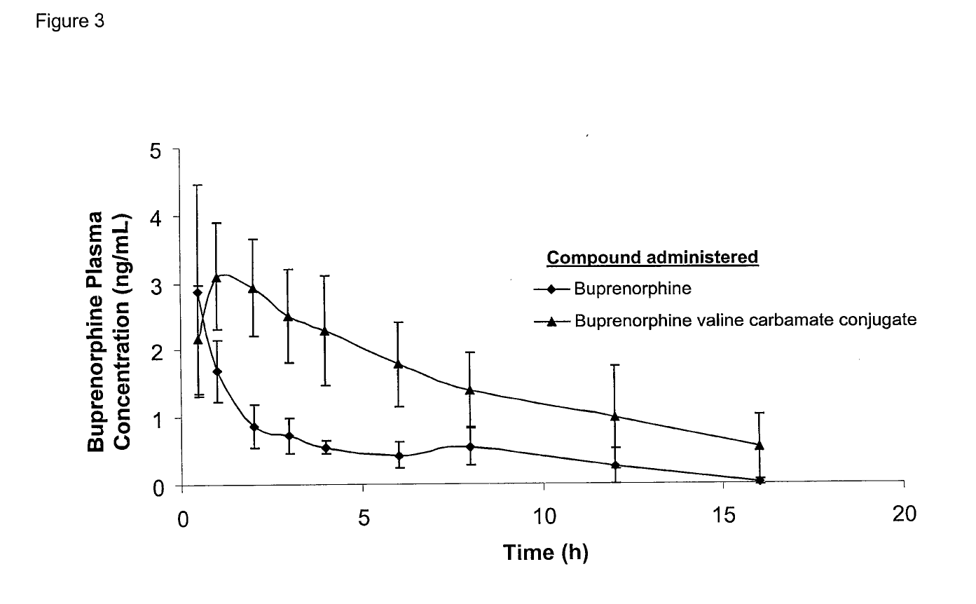

Tables 13 and 14 and FIG. 3 present more detail on PK of buprenorphine after oral administration of either buprenorphine itself or its valine carbamate conjugate.

While peak plasma levels of buprenorphine following the administration of the prodrug were only of the same order as those after giving the parent drug, the overall mean exposure, as reflected in the AUC, was considerably higher being some seven to nine fold greater. This increased exposure was likely a consequence of the sustainment of plasma drugs levels probably resulting from the continuing generation of active drug from the circulating plasma prodrug, concentrations of which, exceeded those of the parent drug by 100-fold. A measure of the sustainment of plasma drug concentrations is given by the period for which such concentrations remained above 50% of the Cₘₐₓ values. After administration of buprenorphine itself this value was 0.5 h compared to 5.0 h after giving the prodrug, providing clear evidence of prolongation of plasma drug levels. This successful maintenance of plasma drug concentrations after giving the prodrug, should ensure less frequent dosing and better sustainment of analgesia and improved patient/subject compliance.

TABLE 13
Pharmacokinetics of buprenorphine after oral administration of the
drug at 0.5 mg buprenorphine free base equivalents/kg to dogs

| Pharmacokinetic | Dog No. | | | | | | |
|---|---|---|---|---|---|---|---|
| parameter | 1 | 2 | 3 | 4 | 5 | Mean | sd |
| Cₘₐₓ (ng/mL) | 5.03 | 2.48 | 17.5 | 1.25 | 2.76 | 5.80/2.88* | 6.70/1.58* |
| Tₘₐₓ (h) | 0.5 | 0.5 | 0.5 | 0.5 | 0.5 | 0.5^{a} | |
| AUCₜ (ng · h/mL) | 4.66 | 2.99 | 21.7 | 0.885 | 1.87 | 6.42/2.60* | 8.65/1.62* |
| AUC₂₄ (ng · h/mL) | 5.27 | 3.49 | 24.3 | 1.41 | 2.56 | 7.47/3.18* | 9.53/1.63* |
| t½ (h) | b | 1.2 | b | b | b | — | |

^{a}Median value
^{b}Calculated as ln2/mean k
^{c}The regression coefficient was ≦0.95, and the fraction of the variance accounted for was ≦0.90, therefore terminal rate constant not reliably estimated
^{d}Calculated using AUC₂₄ values
*Excluding dog 3

TABLE 14
Pharmacokinetics of buprenorphine and its valine carbamate prodrug in the dog after
oral administration of the prodrug at 0.5 mg buprenorphine free base equivalents/kg

| Pharmacokinetic | Dog No. | | | | | | |
|---|---|---|---|---|---|---|---|
| parameter | 1 | 2 | 3 | 4 | 5 | Mean | sd |
| Buprenorphine | | | | | | | |
| Cₘₐₓ (ng/mL) | 2.74 | 4.28 | 2.80 | 2.76 | 2.84 | 3.08 | 0.67 |
| Tₘₐₓ (h) | 1 | 1 | 2 | 2 | 1 | 1^{a} | |
| AUC (ng · h/mL) | 17.6 | 38.4 | 14.3 | 13.9 | 17.1 | 20.2 | 10.3 |
| AUC₂₄ (ng · h/mL) | 20.1 | 43.1 | 16.8 | 16.2 | 19.6 | 23.2 | 11.3 |
| t½ (h) | c | 8.3 | c | 4.4 | 4.1 | 5.6^{b} | |
| Relative F^{d} (%) | 381 | 1235 | 69.1 | 1149 | 766 | 720/883* | 487/391* |
| Buprenorphine | | | | | | | |
| valine carbamate | | | | | | | |
| Cₘₐₓ (ng/mL) | 265 | 342 | 207 | 301 | 348 | 293 | 58 |
| Tₘₐₓ (h) | 1 | 1 | 1 | 2 | 1 | 1^{a} | |
| AUCₜ (ng · h/mL) | 2090 | 3600 | 1770 | 1790 | 2570 | 2360 | 760 |
| AUC₂₄ (ng · h/mL) | 2310 | 3930 | 1810 | 1810 | 2620 | 2500 | 870 |
| t½ (h) | 6.5 | 6.4 | 4.1 | 4.1 | 4.3 | 4.8^{b} | |

^{a}Median value
^{b}Calculated as ln2/mean k
^{c}The regression coefficient was ≦0.95, and the fraction of the variance accounted for was ≦0.90, therefore terminal rate constant not reliably estimated
^{d}Calculated using AUC₂₄ values
*Excluding dog 3

Patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Nothing in this specification should be considered as limiting the scope of the present invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A compound of the formula: 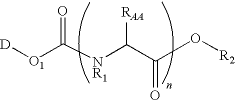
or a pharmaceutically acceptable salt thereof,
wherein,
D is a phenolic analgesic having a low bioavailability,
R₁ and R₂ are independently selected from hydrogen, alkyl, substituted alkyl, cycloalkyl or substituted cycloalkyl group,
R_{AA} is a natural or non-natural amino acid side chain, and each occurrence of R_{AA} and R₁ can be the same or different;
O₁ is an oxygen atom present in the phenolic analgesic; and
n is an integer from 1 to 9,
wherein the compound has an oral bioavailability of at least twice the oral bioavailability of the phenolic analgesic, when administered alone.

2. A meptazinol carbamate prodrug having the formula: 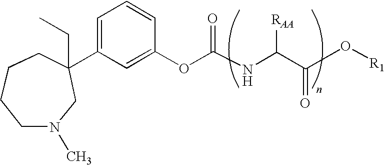
or a pharmaceutically acceptable salt thereof,
wherein,
R₁ is selected from H, an unsubstituted alkyl group and a substituted alkyl group,
n is an integer from 1 to 9;
R_{AA} is a natural or non-natural amino acid side chain; and each occurrence of R_{AA} can be the same or different;

3. An oxymorphone prodrug of the formula: 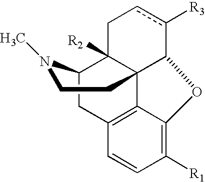
or a pharmaceutically acceptable salt thereof,
wherein,
R₁ and R₂ are selected from 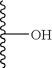
and 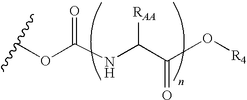
R₃ is selected from 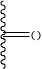
and 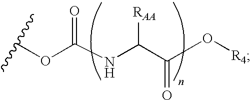
the dashed line in Formula II is absent when R₃ is 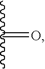
and a bond when R₃ is not 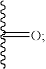
R₄ is independently selected from hydrogen, a substituted alkyl group and an unsubstituted alkyl group;
R_{AA} is a natural or non-natural amino acid side chain, and each occurrence of R_{AA} can be the same or different;
n is an integer selected from 1 to 9 and each occurrence of n can be the same or different;
and at least one of R₁, R₂, and R₃ is

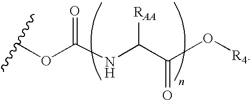

4. A buprenorphine prodrug of the formula: 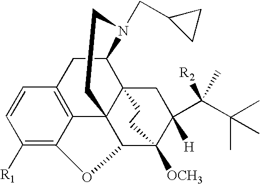
or a pharmaceutically acceptable salt thereof,
wherein,
R₁ and R₂ are selected from 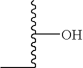
and 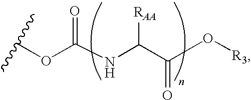
n is an integer from 1 to 9 and each occurrence of n can be the same or different.
R_{AA} is a natural or non-natural amino acid side chain and each occurrence of R_{AA} can be the same or different;
R₃ is H, an unsubstituted alkyl group, or a substituted alkyl group,
and at least one of R₁ and R₂ is

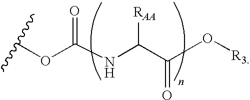

5. A compound selected from the group consisting of meptazinol val carbamate, meptazinol ile carbamate, meptazinol met carbamate, meptazinol tyr carbamate, meptazinol leu carbamate, meptazinol phe carbamate, meptazinol gly carbamate, meptazinol ala-ala carbamate, meptazinol trp carbamate, meptazinol val-ala carbamate, meptazinol asp carbamate, meptazinol val-gly carbamate, meptazinol val-val carbamate, oxymorphone gly carbamate, oxymorphone tyr carbamate, oxymorphone val carbamate, oxymorphone phe carbamate, oxymorphone ile carbamate, oxymorphone met carbamate, buprenorphine leu carbamate, buprenorphine ilr carbamate, buprenorphine lys carbamate, buprenorphine gly carbamate, buprenorphine val carbamate, buprenorphine phe carbamate and buprenorphine asp carbamate, or a pharmaceutically acceptable salt thereof.

6. A method treating pain in a subject in need thereof comprising orally administering a phenolic analgesic carbamate prodrug or pharmaceutically acceptable salt thereof to the subject, wherein the phenolic analgesic carbamate prodrug is comprised of an phenolic analgesic covalently bonded through a carbamate linkage to an amino acid or peptide of 2-9 amino acids in length.

7. The method of claim 6, wherein the pain is selected from nociceptive pain and neuropathic pain.

8. A method for increasing the oral bioavailability of a phenolic analgesic in a subject in need thereof comprising orally administering a phenolic analgesic carbamate prodrug or pharmaceutically acceptable salt thereof to the subject, wherein the phenolic analgesic carbamate prodrug is comprised of an phenolic analgesic covalently bonded through a carbamate linkage to an amino acid or peptide of 2-9 amino acids in length.

9. The method of claim 8, wherein the phenolic analgesic is meptazinol.

10. The method of claim 8, wherein the phenolic analgesic is oxymorphone.

11. The method of claim 8, wherein the phenolic analgesic is buprenorphine.

12. The method of claim 8, wherein the peptide is valine carbamate, L-methionine carbamate, 2-amino-butyric acid carbamate, alanine carbamate, phenylalanine carbamate, isoleucine carbamate, 2-amino acetic acid carbamate, leucine carbamate, isoleucine carbamate, valine-valine carbamate, tyrosine-glycine-carbamate, valine-tyrosine carbamate, tyrosine-valine carbamate, or valine-glycine carbamate.

13. The method of claim 8, wherein the phenolic analgesic carbamate prodrug is selected from the group consisting of meptazinol-(S)-ile carbamate, meptazinol-(S)-leu carbamate, meptazinol-(S)-asp carbamate, meptazinol-(S)-met carbamate, meptazinol-(S)-his carbamate, meptazinol-(S)-phe carbamate, meptazinol-(S)-ser carbamate, meptazinol valine carbamate, meptazinol isoleucine carbamate, meptazinol methionine carbamate, meptazinol alanine carbamate, meptazinol-2-amino-butyric acid carbamate, meptazinol-L-methionine carbamate, and meptazinol glycyl-2-amino acetic acid carbamate, meptazinol-valine-valine carbamate, meptazinol-valine-glycine carbamate, meptazinol-valine-alanine carbamate, meptazinol-tyrosine-valine carbamate, meptazinol-tyrosine-glycine-carbamate, and meptazinol-valine-tyrosine carbamate.

14. The method of claim 8, wherein the phenolic analgesic carbamate prodrug is selected from the group consisting of oxymorphone-S-ile carbamate, oxymorphone-S-leu carbamate, oxymorphone-S-asp carbamate, oxymorphone-S-met carbamate, oxymorphone-S-his carbamate, oxymorphone-S-phe carbamate and oxymorphone-S-ser carbamate, oxymorphone valine carbamate, oxymorphone isoleucine carbamate, oxymorphone methionine carbamate, oxymorphone-valine-valine carbamate, oxymorphone-valine-methionine carbamate, and oxymorphone-valine-isoleucine carbamate.

15. The method of claim 8, wherein the phenolic analgesic carbamate prodrug is selected from the group consisting of meptazinol val carbamate, meptazinol ile carbamate, meptazinol met carbamate, meptazinol tyr carbamate, meptazinol leu carbamate, meptazinol phe carbamate, meptazinol gly carbamate, meptazinol ala-ala carbamate, meptazinol trp carbamate, meptazinol val-ala carbamate, meptazinol asp carbamate, meptazinol val-gly carbamate, meptazinol val-val carbamate, oxymorphone gly carbamate, oxymorphone tyr carbamate, oxymorphone val carbamate, oxymorphone phe carbamate, oxymorphone ile carbamate, oxymorphone met carbamate, buprenorphine leu carbamate, buprenorphine ilr carbamate, buprenorphine lys carbamate, buprenorphine gly carbamate, buprenorphine val carbamate, buprenorphine phe carbamate and buprenorphine asp carbamate or a pharmaceutically acceptable salt thereof.
